SEQUENCE LISTING (201 sequences)

SEQ ID NO: 1 - protein, 6 aa, Artificial
  Synthetic construct
CHFRWC

SEQ ID NO: 2 - protein, 9 aa, Artificial
  Synthetic construct
XRCAHFRWC

SEQ ID NO: 3 - protein, 9 aa, Artificial
  Synthetic construct
YRCAHFRWC

SEQ ID NO: 4 - protein, 9 aa, Artificial
  Synthetic construct
YRCRHFRWC

SEQ ID NO: 5 - protein, 9 aa, Artificial
  Synthetic construct
YRCNHFRWC

SEQ ID NO: 6 - protein, 7 aa, Artificial
  Synthetic construct
CDHFRWC

SEQ ID NO: 7 - protein, 9 aa, Artificial
  Synthetic construct
YRCDHFRWC

SEQ ID NO: 8 - protein, 7 aa, Artificial
  Synthetic construct
CQHFRWC

SEQ ID NO: 9 - protein, 9 aa, Artificial
  Synthetic construct
YRCQHFRWC

SEQ ID NO: 10 - protein, 9 aa, Artificial
  Synthetic construct
YRCQHFRWC

SEQ ID NO: 11 - protein, 9 aa, Artificial
  Synthetic construct
YRCGHFRWC

SEQ ID NO: 12 - protein, 9 aa, Artificial
  Synthetic construct
YRCGHFRWC

SEQ ID NO: 13 - protein, 9 aa, Artificial
  Synthetic construct
YRCHHFRWC

SEQ ID NO: 14 - protein, 9 aa, Artificial
  Synthetic construct
YRCIHFRWC

SEQ ID NO: 15 - protein, 7 aa, Artificial
  Synthetic construct
CLHFRWC

SEQ ID NO: 16 - protein, 7 aa, Artificial
  Synthetic construct
CKHFRWC

SEQ ID NO: 17 - protein, 9 aa, Artificial
  Synthetic construct
YRCMHFRWC

SEQ ID NO: 18 - protein, 9 aa, Artificial
  Synthetic construct
YRCMHFRWC

SEQ ID NO: 19 - protein, 9 aa, Artificial
  Synthetic construct
YRCFHFRWC

SEQ ID NO: 20 - protein, 9 aa, Artificial
  Synthetic construct
YRCPHFRWC

SEQ ID NO: 21 - protein, 9 aa, Artificial
  Synthetic construct
YRCSHFRWC

SEQ ID NO: 22 - protein, 9 aa, Artificial
  Synthetic construct
YRCTHFRWC

SEQ ID NO: 23 - protein, 9 aa, Artificial
  Synthetic construct
YRCWHFRWC

SEQ ID NO: 24 - protein, 9 aa, Artificial
  Synthetic construct
YRCYHFRWC

SEQ ID NO: 25 - protein, 9 aa, Artificial
  Synthetic construct
YRCVHFRWC

SEQ ID NO: 26 - protein, 8 aa, Artificial
  Synthetic construct
RCXHFRWC

SEQ ID NO: 27 - protein, 8 aa, Artificial
  Synthetic construct
RCXHFRWC

SEQ ID NO: 28 - protein, 9 aa, Artificial
  Synthetic construct
YRCXHFRWC

SEQ ID NO: 29 - protein, 7 aa, Artificial
  Synthetic construct
CEHFRWC

SEQ ID NO: 30 - protein, 7 aa, Artificial
  Synthetic construct
CEHFRWC

SEQ ID NO: 31 - protein, 7 aa, Artificial
  Synthetic construct
CEHFRWC

SEQ ID NO: 32 - protein, 7 aa, Artificial
  Synthetic construct
CEHFRWC

SEQ ID NO: 33 - protein, 7 aa, Artificial
  Synthetic construct
CEHFRWC

SEQ ID NO: 34 - protein, 7 aa, Artificial
  Synthetic construct
CEHFRWC

SEQ ID NO: 35 - protein, 9 aa, Artificial
  Synthetic construct
CEHFRWCKP

SEQ ID NO: 36 - protein, 9 aa, Artificial
  Synthetic construct
CEHFRWCSP

SEQ ID NO: 37 - protein, 7 aa, Artificial
  Synthetic construct
CEHFRWC

SEQ ID NO: 38 - protein, 7 aa, Artificial
  Synthetic construct
CEHFRWC

SEQ ID NO: 39 - protein, 7 aa, Artificial
  Synthetic construct
CEHFRWC

SEQ ID NO: 40 - protein, 7 aa, Artificial
  Synthetic construct
CEHFRWC

SEQ ID NO: 41 - protein, 7 aa, Artificial
  Synthetic construct
CEHFRWC

SEQ ID NO: 42 - protein, 7 aa, Artificial
  Synthetic construct
CEHFRWC

SEQ ID NO: 43 - protein, 7 aa, Artificial
  Synthetic construct
CEHFRWC

SEQ ID NO: 44 - protein, 7 aa, Artificial
  Synthetic construct
CEHFRWC

SEQ ID NO: 45 - protein, 8 aa, Artificial
  Synthetic construct
RCEHFRWC

SEQ ID NO: 46 - protein, 8 aa, Artificial
  Synthetic construct
RCEHFRWC

SEQ ID NO: 47 - protein, 8 aa, Artificial
  Synthetic construct
RCEHFRWC

SEQ ID NO: 48 - protein, 8 aa, Artificial
  Synthetic construct
RCEHFRWC

SEQ ID NO: 49 - protein, 8 aa, Artificial
  Synthetic construct
RCEHFRWC

SEQ ID NO: 50 - protein, 8 aa, Artificial
  Synthetic construct
RCEHFRWC

SEQ ID NO: 51 - protein, 8 aa, Artificial
  Synthetic construct
RCEHFRWC

SEQ ID NO: 52 - protein, 8 aa, Artificial
  Synthetic construct
RCEHFRWC

SEQ ID NO: 53 - protein, 8 aa, Artificial
  Synthetic construct
RCEHFRWC

SEQ ID NO: 54 - protein, 8 aa, Artificial
  Synthetic construct
XCEHFRWC

SEQ ID NO: 55 - protein, 8 aa, Artificial
  Synthetic construct
XCEHFRWC

SEQ ID NO: 56 - protein, 8 aa, Artificial
  Synthetic construct
XCEHFRWC

SEQ ID NO: 57 - protein, 8 aa, Artificial
  Synthetic construct
LCEHFRWC

SEQ ID NO: 58 - protein, 8 aa, Artificial
  Synthetic construct
KCEHFRWC

SEQ ID NO: 59 - protein, 8 aa, Artificial
  Synthetic construct
XCEHFRWC

SEQ ID NO: 60 - protein, 8 aa, Artificial
  Synthetic construct
XCEHFRWC

SEQ ID NO: 61 - protein, 10 aa, Artificial
  Synthetic construct
XCEHFRWCSP

SEQ ID NO: 62 - protein, 8 aa, Artificial
  Synthetic construct
XCEHFRWC

SEQ ID NO: 63 - protein, 8 aa, Artificial
  Synthetic construct
VCEHFRWC

SEQ ID NO: 64 - protein, 8 aa, Artificial
  Synthetic construct
RCEHFRWC

SEQ ID NO: 65 - protein, 8 aa, Artificial
  Synthetic construct
RCEHFRWC

SEQ ID NO: 66 - protein, 8 aa, Artificial
  Synthetic construct
RCEHFRWC

SEQ ID NO: 67 - protein, 8 aa, Artificial
  Synthetic construct
RCEHFRWC

SEQ ID NO: 68 - protein, 9 aa, Artificial
  Synthetic construct
YRCEHFRWC

SEQ ID NO: 69 - protein, 9 aa, Artificial
  Synthetic construct
YRCEHFRWC

SEQ ID NO: 70 - protein, 9 aa, Artificial
  Synthetic construct
YRCEHFRWC

SEQ ID NO: 71 - protein, 10 aa, Artificial
  Synthetic construct
YRCEHFRWCE

SEQ ID NO: 72 - protein, 9 aa, Artificial
  Synthetic construct
YRCEHFRWC

SEQ ID NO: 73 - protein, 9 aa, Artificial
  Synthetic construct
YRCEHFRWC

SEQ ID NO: 74 - protein, 9 aa, Artificial
  Synthetic construct
YRCEHFRWC

SEQ ID NO: 75 - protein, 9 aa, Artificial
  Synthetic construct
YRCEHFRWC

SEQ ID NO: 76 - protein, 9 aa, Artificial
  Synthetic construct
YRCEHFRWC

SEQ ID NO: 77 - protein, 9 aa, Artificial
  Synthetic construct
YRCEHFRWC

SEQ ID NO: 78 - protein, 9 aa, Artificial
  Synthetic construct
YRCEHFRWX

SEQ ID NO: 79 - protein, 9 aa, Artificial
  Synthetic construct
YRCEHFRWC

SEQ ID NO: 80 - protein, 9 aa, Artificial
  Synthetic construct
YRCEHFRWC

SEQ ID NO: 81 - protein, 9 aa, Artificial
  Synthetic construct
YRCEHFRWC

SEQ ID NO: 82 - protein, 9 aa, Artificial
  Synthetic construct
YRCEHFRWC

SEQ ID NO: 83 - protein, 9 aa, Artificial
  Synthetic construct
YRCEHFRWC

SEQ ID NO: 84 - protein, 9 aa, Artificial
  Synthetic construct
YRCEHFRWC

SEQ ID NO: 85 - protein, 9 aa, Artificial
  Synthetic construct
YRCEHFRWC

SEQ ID NO: 86 - protein, 9 aa, Artificial
  Synthetic construct
YRCEHFRWC

SEQ ID NO: 87 - protein, 8 aa, Artificial
  Synthetic construct
RCEHFRWC

SEQ ID NO: 88 - protein, 9 aa, Artificial
  Synthetic construct
YRCEHFRWC

SEQ ID NO: 89 - protein, 9 aa, Artificial
  Synthetic construct
YRCEHFRWC

SEQ ID NO: 90 - protein, 9 aa, Artificial
  Synthetic construct
YRCEHFRWC

SEQ ID NO: 91 - protein, 9 aa, Artificial
  Synthetic construct
YRCEHFRWC

SEQ ID NO: 92 - protein, 9 aa, Artificial
  Synthetic construct
YRCEHFRWC

SEQ ID NO: 93 - protein, 9 aa, Artificial
  Synthetic construct
YRCEHFRWC

SEQ ID NO: 94 - protein, 9 aa, Artificial
  Synthetic construct
YRCEHFRWC

SEQ ID NO: 95 - protein, 9 aa, Artificial
  Synthetic construct
YRCEHFRWC

SEQ ID NO: 96 - protein, 9 aa, Artificial
  Synthetic construct
YRCEHFRWC

SEQ ID NO: 97 - protein, 9 aa, Artificial
  Synthetic construct
YRCEHFRWC

SEQ ID NO: 98 - protein, 9 aa, Artificial
  Synthetic construct
YRCEHFRWC

SEQ ID NO: 99 - protein, 9 aa, Artificial
  Synthetic construct
YRCEHFRWC

SEQ ID NO: 100 - protein, 9 aa, Artificial
  Synthetic construct
YRCEHFRWC

SEQ ID NO: 101 - protein, 9 aa, Artificial
  Synthetic construct
YRCEHFRWC

SEQ ID NO: 102 - protein, 9 aa, Artificial
  Synthetic construct
YRCEAFRWC

SEQ ID NO: 103 - protein, 9 aa, Artificial
  Synthetic construct
YRCEAFRWC

SEQ ID NO: 104 - protein, 9 aa, Artificial
  Synthetic construct
YRCEAFRWC

SEQ ID NO: 105 - protein, 9 aa, Artificial
  Synthetic construct
YRCEAFRWC

SEQ ID NO: 106 - protein, 9 aa, Artificial
  Synthetic construct
YRCEAFRWC

SEQ ID NO: 107 - protein, 9 aa, Artificial
  Synthetic construct
YRCEAFRWC

SEQ ID NO: 108 - protein, 9 aa, Artificial
  Synthetic construct
YRCEAFRWC

SEQ ID NO: 109 - protein, 9 aa, Artificial
  Synthetic construct
YRCEAFRWC

SEQ ID NO: 110 - protein, 9 aa, Artificial
  Synthetic construct
YRCEAFRWC

SEQ ID NO: 111 - protein, 9 aa, Artificial
  Synthetic construct
YRCEAFRWC

SEQ ID NO: 112 - protein, 9 aa, Artificial
  Synthetic construct
YRCEAFRWC

SEQ ID NO: 113 - protein, 9 aa, Artificial
  Synthetic construct
YRCEAFRWC

SEQ ID NO: 114 - protein, 9 aa, Artificial
  Synthetic construct
YRCEHFRWC

SEQ ID NO: 115 - protein, 9 aa, Artificial
  Synthetic construct
YRCEHFRWC

SEQ ID NO: 116 - protein, 10 aa, Artificial
  Synthetic construct
YRCEHFRWCX

SEQ ID NO: 117 - protein, 9 aa, Artificial
  Synthetic construct
YRCEHFRWC

SEQ ID NO: 118 - protein, 10 aa, Artificial
  Synthetic construct
YRCEHFRWCE

SEQ ID NO: 119 - protein, 11 aa, Artificial
  Synthetic construct
YRCEHFRWCSP

SEQ ID NO: 120 - protein, 11 aa, Artificial
  Synthetic construct
YRCEHFRWCSX

SEQ ID NO: 121 - protein, 11 aa, Artificial
  Synthetic construct
YRCEHFRWCKP

SEQ ID NO: 122 - protein, 11 aa, Artificial
  Synthetic construct
YRCEHFRWCKX

SEQ ID NO: 123 - protein, 11 aa, Artificial
  Synthetic construct
YRCEHFRWCRF

SEQ ID NO: 124 - protein, 9 aa, Artificial
  Synthetic construct
YXCEHFRWC

SEQ ID NO: 125 - protein, 9 aa, Artificial
  Synthetic construct
YXCEHFRWC

SEQ ID NO: 126 - protein, 9 aa, Artificial
  Synthetic construct
YXCEHFRWC

SEQ ID NO: 127 - protein, 9 aa, Artificial
  Synthetic construct
YXCEHFRWC

SEQ ID NO: 128 - protein, 9 aa, Artificial
  Synthetic construct
YKCEHFRWC

SEQ ID NO: 129 - protein, 9 aa, Artificial
  Synthetic construct
YSCEHFRWC

SEQ ID NO: 130 - protein, 9 aa, Artificial
  Synthetic construct
YVCEHFRWC

SEQ ID NO: 131 - protein, 9 aa, Artificial
  Synthetic construct
YRCEHFRWC

SEQ ID NO: 132 - protein, 6 aa, Artificial
  Synthetic construct
XHFRWC

SEQ ID NO: 133 - protein, 6 aa, Artificial
  Synthetic construct
XHFRWC

SEQ ID NO: 134 - protein, 6 aa, Artificial
  Synthetic construct
XHFRWC

SEQ ID NO: 135 - protein, 6 aa, Artificial
  Synthetic construct
XHFRWC

SEQ ID NO: 136 - protein, 6 aa, Artificial
  Synthetic construct
XHFRWC

SEQ ID NO: 137 - protein, 6 aa, Artificial
  Synthetic construct
XHFRWC

SEQ ID NO: 138 - protein, 6 aa, Artificial
  Synthetic construct
XHFRWC

SEQ ID NO: 139 - protein, 6 aa, Artificial
  Synthetic construct
XHFRWC

SEQ ID NO: 140 - protein, 6 aa, Artificial
  Synthetic construct
XHFRWC

SEQ ID NO: 141 - protein, 6 aa, Artificial
  Synthetic construct
XHFRWC

SEQ ID NO: 142 - protein, 6 aa, Artificial
  Synthetic construct
XHFRWC

SEQ ID NO: 143 - protein, 6 aa, Artificial
  Synthetic construct
XHFRWC

SEQ ID NO: 144 - protein, 6 aa, Artificial
  Synthetic construct
XHFRWC

SEQ ID NO: 145 - protein, 6 aa, Artificial
  Synthetic construct
XHFRWC

SEQ ID NO: 146 - protein, 6 aa, Artificial
  Synthetic construct
XHFRWC

SEQ ID NO: 147 - protein, 6 aa, Artificial
  Synthetic construct
XHFRWC

SEQ ID NO: 148 - protein, 6 aa, Artificial
  Synthetic construct
XHFRWC

SEQ ID NO: 149 - protein, 6 aa, Artificial
  Synthetic construct
XHFRWC

SEQ ID NO: 150 - protein, 6 aa, Artificial
  Synthetic construct
XHFRWC

SEQ ID NO: 151 - protein, 6 aa, Artificial
  Synthetic construct
XHFRWC

SEQ ID NO: 152 - protein, 6 aa, Artificial
  Synthetic construct
XHFRWC

SEQ ID NO: 153 - protein, 7 aa, Artificial
  Synthetic construct
RXHFRWC

SEQ ID NO: 154 - protein, 7 aa, Artificial
  Synthetic construct
RXHFRWC

SEQ ID NO: 155 - protein, 7 aa, Artificial
  Synthetic construct
RXHFRWC

SEQ ID NO: 156 - protein, 7 aa, Artificial
  Synthetic construct
RXHFRWC

SEQ ID NO: 157 - protein, 7 aa, Artificial
  Synthetic construct
RXHFRWC

SEQ ID NO: 158 - protein, 7 aa, Artificial
  Synthetic construct
RXHFRWC

SEQ ID NO: 159 - protein, 7 aa, Artificial
  Synthetic construct
RXHFRWC

SEQ ID NO: 160 - protein, 7 aa, Artificial
  Synthetic construct
XXHFRWC

SEQ ID NO: 161 - protein, 7 aa, Artificial
  Synthetic construct
GXHFRWC

SEQ ID NO: 162 - protein, 8 aa, Artificial
  Synthetic construct
YRXHFRWC

SEQ ID NO: 163 - protein, 8 aa, Artificial
  Synthetic construct
YRXHFRWC

SEQ ID NO: 164 - protein, 8 aa, Artificial
  Synthetic construct
YRXHFRWC

SEQ ID NO: 165 - protein, 8 aa, Artificial
  Synthetic construct
YRXHFRWC

SEQ ID NO: 166 - protein, 9 aa, Artificial
  Synthetic construct
YRXEHFRWC

SEQ ID NO: 167 - protein, 6 aa, Artificial
  Synthetic construct
XHARWC

SEQ ID NO: 168 - protein, 6 aa, Artificial
  Synthetic construct
XHFRWX

SEQ ID NO: 169 - protein, 6 aa, Artificial
  Synthetic construct
XHFRWX

SEQ ID NO: 170 - protein, 6 aa, Artificial
  Synthetic construct
XHFRWX

SEQ ID NO: 171 - protein, 6 aa, Artificial
  Synthetic construct
XHFRWX

SEQ ID NO: 172 - protein, 6 aa, Artificial
  Synthetic construct
XHFRWX

SEQ ID NO: 173 - protein, 6 aa, Artificial
  Synthetic construct
XHFRWX

SEQ ID NO: 174 - protein, 6 aa, Artificial
  Synthetic construct
XHFRWX

SEQ ID NO: 175 - protein, 6 aa, Artificial
  Synthetic construct
XHFRWX

SEQ ID NO: 176 - protein, 6 aa, Artificial
  Synthetic construct
XHFRWX

SEQ ID NO: 177 - protein, 7 aa, Artificial
  Synthetic construct
XXHFRWX

SEQ ID NO: 178 - protein, 7 aa, Artificial
  Synthetic construct
GXHFRWX

SEQ ID NO: 179 - protein, 6 aa, Artificial
  Synthetic construct
XHFRWX

SEQ ID NO: 180 - protein, 6 aa, Artificial
  Synthetic construct
CHFRWX

SEQ ID NO: 181 - protein, 6 aa, Artificial
  Synthetic construct
CHFRWX

SEQ ID NO: 182 - protein, 6 aa, Artificial
  Synthetic construct
CHFRWX

SEQ ID NO: 183 - protein, 6 aa, Artificial
  Synthetic construct
CHFRWX

SEQ ID NO: 184 - protein, 6 aa, Artificial
  Synthetic construct
CHFRWX

SEQ ID NO: 185 - protein, 6 aa, Artificial
  Synthetic construct
CHFRWX

SEQ ID NO: 186 - protein, 7 aa, Artificial
  Synthetic construct
RCHFRWX

SEQ ID NO: 187 - protein, 7 aa, Artificial
  Synthetic construct
RCHFRWX

SEQ ID NO: 188 - protein, 7 aa, Artificial
  Synthetic construct
RCHFRWX

SEQ ID NO: 189 - protein, 7 aa, Artificial
  Synthetic construct
RCHFRWX

SEQ ID NO: 190 - protein, 7 aa, Artificial
  Synthetic construct
RCHFRWX

SEQ ID NO: 191 - protein, 7 aa, Artificial
  Synthetic construct
RCHFRWX

SEQ ID NO: 192 - protein, 9 aa, Artificial
  Synthetic construct
YRCEHFRWX

SEQ ID NO: 193 - protein, 6 aa, Artificial
  Synthetic construct
XHFRWX

SEQ ID NO: 194 - protein, 7 aa, Artificial
  Synthetic construct
RXHFRWX

SEQ ID NO: 195 - protein, 7 aa, Artificial
  Synthetic construct
RXHFRWX

SEQ ID NO: 196 - protein, 8 aa, Artificial
  Synthetic construct
YRXHFRWX

SEQ ID NO: 197 - protein, 9 aa, Artificial
  Synthetic construct
YRXEHFRWX

SEQ ID NO: 198 - protein, 6 aa, Artificial
  Synthetic construct
CHFRWC

SEQ ID NO: 199 - protein, 9 aa, Artificial
  Synthetic construct
XXXXXXWXX

SEQ ID NO: 200 - protein, 9 aa, Artificial
  Synthetic construct
XXXHFRWXX

SEQ ID NO: 201 - protein, 9 aa, Artificial
  Synthetic construct
XXXHFRWXX

(19) United States
(12) Patent Application Publication
(10) Pub. No.: US 2006/0293223 A1
(43) Pub. Date: Dec. 28, 2006

(54) Uses of melanocortin-3 receptor (mc3r) agonist peptides

(75) Inventors: Robert Alan Gadski, Indianapolis, IN (US); Mark Louis Heiman, Indianapolis, IN (US); Hansen Maxwell Hsiung, Indianapolis, IN (US); John P. Mayer, Indianapolis, IN (US); Liang Zeng Yan, Carmel, IN (US)

Correspondence Address: ELI LILLY & COMPANY, PATENT DIVISION, P.O. BOX 6288, INDIANAPOLIS, IN, 46206-6288, US

(73) Assignee: Eli Lilly and Company Patent Division, Indianapolis, IN (US)

(21) Appl. No.: 10/556,690

(22) PCT Filed: Jun. 17, 2004
(86) PCT No.: PCT/US04/16624

§ 371 Date: Nov. 15, 2005

Related U.S. Application Data
(60) Provisional application No. 60/479,740, filed on Jun. 19, 2003.
(60) Provisional application No. 60/570,737, filed on May 13, 2004.

(51) Int. Cl. A61K 38/12

(52) U.S. Cl. 514/9

(57) ABSTRACT

The present invention provides methods of treating metabolic disorders, including, for example, obesity, diabetes mellitus, cachexia, sarcopenia, and cardiovascular disorders, methods of inducing weight loss and increasing muscle mass in a patient, by administration of MC3R agonist peptides. Furthermore, the present invention provides the use of an MC3R agonist peptide for the manufacture of a medicament for the treatment of metabolic disorders, including, for example, obesity, diabetes mellitus, cachexia, sarcopenia, and cardiovascular disorders, inducing weight loss and increasing muscle mass in a patient.

The present invention relates to uses of peptide agonists of the MC3 receptor in the treatment of disorders responsive to the activation of this receptor, such as obesity, diabetes mellitus, and cachexia.

The proopiomelanocortin (POMC) gene encodes a 31-36 kDa pre-prohormone, from which seven mature peptide hormones are derived. POMC processing occurs in a tissue specific manner yielding four distinct melanocortin peptides: adrenocorticotropic hormone (ACTH), α-melanocyte stimulating hormone (α-MSH), β-MSH, and γ-MSH.

Five melanocortin receptors have thus far been identified and are referred to herein as MC1, MC2, MC3, MC4, and MC5. MC1, whose primary endogenous ligand is α-MSH, is associated with pigmentation. MC2, whose primary endogenous-ligand is ACTH, is associated with steroidogenesis. MC2 is distinctly different from the other melanocortin receptors and is not expected to interact with endogenous or synthetic MSHs other than ACTH or analogues thereof (Schiöth et al., Life Sciences 59(10):797-801, 1996). MC5 is believed to have two primary ligands, α-MSH and ACTH, and is associated with exocrine Amenand sebaceous gland lipid secretion.

MacNeil et al. provide an excellent overview of the melanocortins and their function relating to body weight regulation (Eur. J. Pharm. 440(2-3): 141-57, 2002). Both MC3 and MC4 are expressed in the brain. MC4 is expressed throughout the brain, whereas MC3 is expressed predominantly in the hypothalamus, leading to an abundance of MC3 there (Roselli-Rehfuss et al., PNAS USA 90:8856-60, 1993). Both MC3 and MC4 are involved in regulating energy metabolism. Analysis of Mc3r−/− mice suggests that the MC3 receptor is complementary to the MC4 receptor's role in regulating body weight. The mice, although not significantly overweight, exhibit increased adiposity, with an increased feeding efficiency (Butler et al., Endocrinol. 141:3518-21, 2000; Chen et al., Nat. Genet. 26:97-102, 2000). Kim et al. suggest that the MC4 receptor regulates food intake and energy expenditure, whereas MC3 receptor regulates feeding efficiency and partitioning of nutrients into fat (Diabetes 49:177-82, 2000; J. Clin. Invest. 105(7): 1005-11, 2000). Furthermore, both receptors regulate insulin activity (Fan et al., Endocrinol 141(9):3072-79, 2000; Obici et al., J. Clin. Invest. 108:1079-85, 2001).

Additionally, MC3 receptor is expressed in peripheral tissues such as heart, gut, stomach, pancreas, placenta, testis, ovary, muscle, and kidney (Gantz et al., J. Biol. Chem. 268:8246-50, 1993; Chhajlani, Biochem. Mol. Biol. Int. 38:73-80, 1996). Gamma-melanocortin stimulating hormone (γ-MSH), a compound that reduces blood pressure and heart rate when administered by intracerebroventricular administration, preferentially activates MC3 receptor. This suggests that the MC3 receptor may play a role in regulating cardiovascular functions (Versteeg et al., Eur. J. Pharmacol 360:1-14, 1998).

The development of selective peptide agonists for melanocortin receptors has. closely followed the identification of the various melanocortin receptor subtypes and their perceived primary ligands. MacNeil, supra. α-MSH, a 13-amino acid peptide, is a non-selective agonist at four melanocortin receptors, MC1 and MC3-MC5. NDP-αMSH is a more potent, protease resistant, but still non-selective analogue of α-MSH.

The lactam derived from the 4-10 fragment of NDP-αMSH, known as MTII, is even more potent in vivo than NDP-αMSH but is non-selective. Replacement of the D-Phe with D-(2′)Nal in MTII, yielded a high affinity antagonist for MC3 and MC4 that is an agonist for the MC1 and MC5 receptors. This peptide is known as SHU9119.

Despite some progress in the field of melanocortin agonist development, a need exists for MC3 agonists with pharmaceutically desirable selectivity, potency, and efficacy, for use as a pharmaceutical, in particular, for the treatment of metabolic disorders, including, for example, obesity, diabetes, cachexia, sarcopenia, and dyslipidemias. Especially desired are MC3 agonists with a clinically desirable pharmacology and safety profile.

Obesity

Obesity, and especially upper body obesity, is a common and very serious public health problem in the United States and throughout the world. According to recent statistics, more than 25% of the United States population and 27% of the Canadian population are overweight. Kuczmarski, Amer. J. of Clin. Nutr. 55:495S-502S, 1992; Reeder et al., Can. Med. Assn. J., 23:226-33, 1992. Upper body obesity is the strongest risk factor known for type II diabetes mellitus, and is a strong risk factor for cardiovascular disease and cancer as well. Recent estimates for the medical cost of obesity are $150,000,000,000 worldwide. The problem has become serious enough that the surgeon general has begun an initiative to combat the ever-increasing adiposity rampant in American society. Because of MC3's complementary role to MC4R in regulating body weight, agonism of the MC3 receptor may be useful for the treatment of obesity.

Diabetes

Diabetes is a disease in which a mammal's ability to regulate glucose levels in the blood is impaired because the mammal has a reduced ability to convert glucose to glycogen for storage in muscle and liver cells. In Type I diabetes, this reduced ability to store glucose is caused by reduced insulin production. “Type II Diabetes” or “non-insulin dependent diabetes mellitus” (NIDDM) is a form of diabetes which is due to a profound resistance to insulin stimulating or regulatory effect on glucose and lipid metabolism in the main insulin-sensitive tissues: muscle, liver, and adipose tissue. This resistance to insulin responsiveness results in insufficient insulin activation of glucose uptake, oxidation, and storage in muscle and inadequate insulin repression of lipolysis in adipose tissue and of glucose production and secretion in liver. When these cells become desensitized to insulin, the body tries to compensate by producing abnormally high levels of insulin, and hyperinsulemia results. Hyperinsulemia is associated with hypertension and elevated body weight. Since insulin is involved in promoting the cellular uptake of glucose, amino acids, and triglycerides from the blood by insulin sensitive cells, insulin insensitivity can result in elevated levels of triglycerides and LDL which are risk factors in cardiovascular diseases. The constellation of symptoms, which includes hyperinsulemia, combined with hypertension, elevated body weight, elevated triglycerides and elevated LDL, is known as Syndrome X. Similar to MC4R, MC3R is known to regulate insulin activity. In light of this, MC3R agonist peptides may be useful for the treatment of diabetes mellitus and related disorders, such as Syndrome X.

Weight Loss and Frailty Disorders

Cachexia is a debilitating condition usually associated with an advanced stage disease such as cancer. The weight loss resulting from cachexia includes loss of fatty tissue as well as lean body mass such as muscle and even bone loss. Additionally, it leads to loss of appetite (anorexia), weakness (asthenia), and anemia. Marks and Cone teach that the MC3 receptor plays a role in the disease cachexia (Ann. NY Acad. Sci. 994:258-66, 2003).

Sarcopenia is age-related loss of muscle. Like other degenerative diseases such as arthritis and osteoporosis, sarcopenia affects body movement and function, increasing the risk of falls and injuries. As muscle mass decreases, frailty increases due to weakening of the bones. Moreover, loss of muscle negatively impacts metabolic function, which can lead to obesity, diabetes, and impaired ability to regulate body temperature. Because of MC3's regulatory role in the partitioning of nutrients into fat, agonism of the MC3 receptor may be useful for the treatment of sarcopenia and frailty disorders.

Dyslipidemias and Cardiovascular Disorders

Dyslipidemias are disorders related to the level of lipids in the blood. Such lipids include low density and high density lipoprotein, and triglycerides. Dyslipidemia and its pathological sequelae, e.g., atherosclerosis, elevated blood pressure, hypertension, stroke, diabetes, kidney disease, hypothyroidism, etc., are a major cause of death, morbidity, and economic loss in the human population. Despite the use of cholesterol-lowering drugs such as statins, hypercholesterolemia and other dyslipidemias still remain a problem. Versteeg et al., supra, suggest that the MC3 receptor may play a role in regulating cardiovascular functions; hence, MC3R agonist peptides may be useful in treating cardiovascular disorders. See, e.g., Ni et al., J. Clin. Invest. 111(8):1251-8, 2003; and Reudelhuber, J. Clin. Invest. 111 (8):1115-6, 2003.

Considering the disorders associated with or regulated by the MC3 receptor, a need exists to provide MC3 agonists useful in treating such disorders. To meet this need, this application provides such MC3 agonist peptides and their methods of use in treating metabolic disorders, inducing weight loss, and increasing muscle mass.

In one embodiment, the present invention relates to a method for agonizing the MC3 receptor, which comprises administering to a patient in need thereof an effective amount of an MC3R agonist peptide as described below (hereinafter “MC3R agonist peptide”).

In another embodiment, the present invention relates to a method of treating obesity in a patient, comprising the step of administering to the patient in need thereof a pharmaceutically effective amount of an MC3R agonist peptide.

In another embodiment, the present invention relates to a method of treating diabetes mellitus in a patient, comprising the step of administering to the patient in need thereof a pharmaceutically effective amount of an MC3R agonist peptide.

In another embodiment, the present invention relates to a method of treating cachexia in a patient, comprising the step of administering to the patient in need thereof a pharmaceutically effective amount of an MC3R agonist peptide.

In another embodiment, the present invention relates to a method of treating sarcopenia in a patient, comprising the step of administering to the patient in need thereof a pharmaceutically effective amount of an MC3R agonist peptide.

In another embodiment, the present invention relates to a method of inducing weight loss in a patient, comprising the step of administering to the patient in need thereof a pharmaceutically effective amount of an MC3R agonist peptide.

In another embodiment, the present invention relates to a method of increasing muscle mass in a patient, comprising the step of administering to the patient in need thereof a pharmaceutically effective amount of an MC3R agonist peptide.

In another embodiment, the present invention relates to a method of treating cardiovascular disorders, such as dyslipidemias and hypertension, in a patient, comprising the step of administering to the patient in need thereof a pharmaceutically effective amount of an MC3R agonist peptide.

In another embodiment, the present invention is further related to the use of the compound of an MC3R agonist peptide as a medicament.

In another embodiment, the present invention is further related to the use of an MC3R agonist peptide in the manufacture of a medicament for the treatment of obesity.

In another embodiment, the present invention is further related to the use of the compound of an MC3R agonist peptide in the manufacture of a medicament for the treatment of diabetes mellitus.

In another embodiment, the present invention is further related to the use of an MC3R agonist peptide in the manufacture of a medicament for the treatment of cachexia.

In another embodiment, the present invention is further related to the use of an MC3R agonist peptide in the manufacture of a medicament for the treatment of sarcopenia.

In another embodiment, the present invention is further related to the use of an MC3R agonist peptide in the manufacture of a medicament for inducing weight loss.

In another embodiment, the present invention is further related to the use of an MC3R agonist peptide in the manufacture of a medicament for increasing muscle mass.

In another embodiment, the present invention is further related to the use of an MC3R agonist peptide in the manufacture of a medicament for the treatment of cardiovascular disorders, such as dyslipidemias and hypertension.

For the purposes of the present invention, as disclosed and claimed herein, the following terms are as defined below.

The following terms are used when referring to a melanocortin receptor: “MCX receptor,” “MCXR,” and “MCX,” wherein the X is a number from 1 to 5 referring to the specific melanocortin receptor. For example, a melanocortin-3 receptor is interchangeably referred to as “MC3 receptor,” “MC3R,” and “MC3,” throughout this specification. In U.S. Provisional Patent Application Nos. 60/479,740 and 60/570,737, a series of MC4R agonist peptides are defined. It has been found that many of these MC4R agonist peptides are also useful as MC3R agonist peptides, which will be defined below. The “MC4R agonist peptides,” defined in the aforementioned applications, include any agonist peptide represented by the following Structural Formula I (SEQ ID NO: 199):

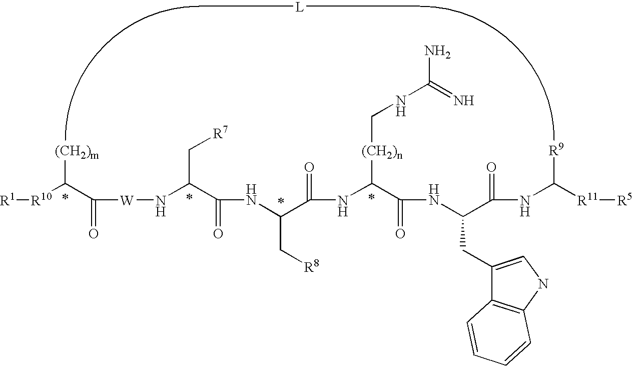

and pharmaceutically acceptable salts thereof, wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH₂)_{q}C(O)—, 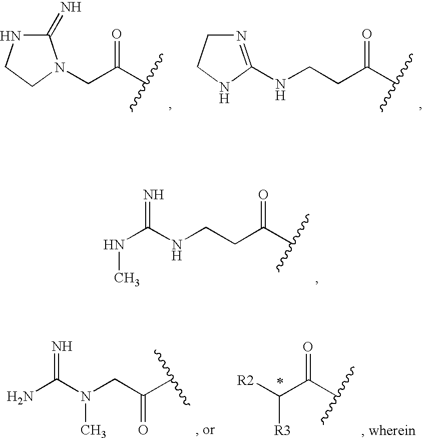
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr-NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile, 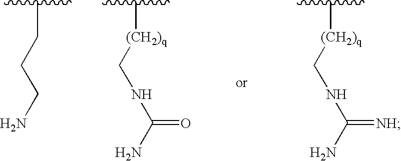
q is 0, 1, 2, or 3;
R⁶ is a phenyl or C₈-C₁₄ bicyclic aryl;
m is 1 or2;
n is 1, 2, 3,or 4;
R⁹ is (CH₂)ₚ or (CH₃)₂C—;
p is 1 or 2;
R¹⁰ is NH— or is absent;
R⁷ is a 5- or 6-membered heteroaryl or a 5- or 6-membered heteroaryl ring optionally substituted with R⁴;
R⁴ is H, C₁-C₄ straight or branched alkyl, phenyl, benzyl, or (C₆H₅)—CH₂—O—CH₂—;
R⁸ is phenyl, a phenyl ring optionally substituted with X, or cyclohexyl;
X is H, Cl, F, Br, methyl, or methoxy;
R¹¹ is —C(O) or —CH₂;
R⁵ is —NH₂, —OH, glycinol, NH₂-Pro-Ser-, NH₂-Pro-Lys-, HO-Ser-, HO-Pro-Ser-, HO-Lys-, -Ser alcohol, -Se-Pro alcohol, -Lys-Pro alcohol, HOCH₂CH₂—O—CH₂CH₂NH—, NH₂-Phe-Arg-, NH₂-Glu-, NH₂CH₂RCH₂NH—, RHN—, or RO— where R is a C¹-C₄ straight or branched alkyl; and
L is —S—S— or —S—CH₂—S—.

MC4R agonist peptides, as defined above, include, but are not limited to, those compounds listed in the following table:

TABLE 1
Specific MC4R agonist peptides.

| No. | Name |
|---|---|
| 1 | Ac-cyclo[Cys-His-D-Phe-Arg-Trp-Cys]-NH₂ |
| 2 | Ac-Cya-Arg-cyclo[Cys-Ala-His-D-Phe-Arg-Trp-Cys]-NH₂ |
| 3 | Ac-Tyr-Arg-cyclo[Cys-Ala-His-D-Phe-Arg-Trp-Cys]-NH₂ |
| 4 | Ac-Tyr-Arg-cyclo[Cys-Arg-His-D-Phe-Arg-Trp-Cys]-NH₂ |
| 5 | Ac-Tyr-Arg-cyclo[Cys-Asn-His-D-Phe-Arg-Trp-Cys]-NH₂ |
| 6 | Ac-cyclo[Cys-Asp-His-D-Phe-Arg-Trp-Cys]-NH₂ |
| 7 | Ac-Tyr-Arg-cyclo[Cys-Asp-His-D-Phe-Arg-Trp-Cys]-NH₂ |
| 8 | Ac-cyclo[Cys-Gln-His-D-Phe-Arg-Trp-Cys]-NH₂ |
| 9 | Ac-Tyr-Arg-cyclo[Cys-Gln-His-D-Phe-Arg-Trp-Cys]-OH |
| 10 | Ac-Tyr-Arg-cyclo[Cys-Gln-His-D-Phe-Arg-Trp-Cys]-OMe |
| 11 | Tyr-Arg-cyclo[Cys-Gly-His-D-Phe-Arg-Trp-Cys]-NH₂ |
| 12 | Ac-Tyr-Arg-cyclo[Cys-Gly-His-D-Phe-Arg-Trp-Cys]-NH₂ |
| 13 | Ac-Tyr-Arg-cyclo[Cys-His-His-D-Phe-Arg-Trp-Cys]-NH₂ |
| 14 | Ac-Tyr-Arg-cyclo[Cys-Ile-His-D-Phe-Arg-Trp-Cys]-NH₂ |
| 15 | Ac-cyclo[Cys-Leu-His-D-Phe-Arg-Trp-Cys]-NH₂ |
| 16 | Ac-cyclo[Cys-Lys-His-D-Phe-Arg-Trp-Cys]-NH₂ |
| 17 | N-methyl-Tyr-Arg-cyclo[Cys-Met-His-D-Phe-Arg-Trp-Cys]-NH₂ |
| 18 | Ac-Tyr-Arg-cyclo[Cys-Met-His-D-Phe-Arg-Trp-Cys]-NH₂ |
| 19 | Ac-Tyr-Arg-cyclo[Cys-Phe-His-D-Phe-Arg-Trp-Cys]-NH₂ |
| 20 | Ac-Tyr-Arg-cyclo[Cys-Pro-His-D-Phe-Arg-Trp-Cys]-NH₂ |
| 21 | Ac-Tyr-Arg-cyclo[Cys-Ser-His-D-Phe-Arg-Trp-Cys]-NH₂ |
| 22 | Ac-Tyr-Arg-cyclo[Cys-Thr-His-D-Phe-Arg-Trp-Cys]-NH₂ |
| 23 | Ac-Tyr-Arg-cyclo[Cys-Trp-His-D-Phe-Arg-Trp-Cys]-NH₂ |
| 24 | Ac-Tyr-Arg-cyclo[Cys-Tyr-His-D-Phe-Arg-Trp-Cys]-NH₂ |
| 25 | Ac-Tyr-Arg-cyclo[Cys-Val-His-D-Phe-Arg-Trp-Cys]-NH₂ |
| 26 | Ac-Arg-cyclo[Cys-Cya-His-D-Phe-Arg-Trp-Cys]-NH₂ |
| 27 | Ac-D-Arg-cyclo[Cys-Cya-His-D-Phe-Arg-Trp-Cys]-NH₂ |
| 28 | Ac-Tyr-Arg-cyclo[Cys-Cya-His-D-Phe-Arg-Trp-Cys]-NH₂ |
| 29 | cyclo[Cys-Glu-His-D-Phe-Arg-Trp-Cys]-NH₂ |
| 30 | Ac-cyclo[Cys-Glu-His-D-Phe-Arg-Trp-Cys]-NH₂ |
| 31 | Ac-cyclo[Cys-Glu-His-(4-F-D-Phe)-Arg-Trp-Cys]-NH₂ |
| 32 | Ac-cyclo[Cys-Glu-His-(4-Cl-D-Phe)-Arg-Trp-Cys]-NH₂ |
| 33 | Ac-cyclo[Cys-Glu-His-(4-Br-D-Phe)-Arg-Trp-Cys]-NH₂ |
| 34 | Ac-cyclo[Cys-Glu-(1-Me-His)-D-Phe-Arg-Trp-Cys]-NH₂ |
| 35 | Ac-cyclo[Cys-Glu-His-D-Phe-Arg-Trp-Cys]-Lys-Pro-NH₂ |
| 36 | Ac-cyclo[Cys-Glu-His-D-Phe-Arg-Trp-Cys]-Ser-Pro-NH₂ |
| 37 | N-propionyl-cyclo[Cys-Glu-His-D-Phe-Arg-Trp-Cys]-NH₂ |
| 38 | N-butyryl-cyclo[Cys-Glu-His-D-Phe-Arg-Trp-Cys]-NH₂ |
| 39 | N-valeryl-cyclo[Cys-Glu-His-D-Phe-Arg-Trp-Cys]-NH₂ |
| 40 | 3-guanidinopropionyl-cyclo[Cys-Glu-His-D-Phe-Arg-Trp-Cys]-NH₂ |
| 41 | 4-guanidinobutyryl-cyclo[Cys-Glu-His-D-Phe-Arg-Trp-Cys]-NH₂ |
| 42 | 5-guanidinovaleryl-cyclo[Cys-Glu-His-D-Phe-Arg-Trp-Cys]-NH₂ |
| 43 | Ac-diaminopropionyl-cyclo[Cys-Glu-His-D-Phe-Arg-Trp-Cys]-NH₂ |
| 44 | Ac-diaminobutyryl-cyclo[Cys-Glu-His-D-Phe-Arg-Trp-Cys]-NH₂ |
| 45 | Arg-cyclo[Cys-Glu-His-D-Phe-Arg-Trp-Cys]-OH |
| 46 | D-Arg-cyclo[Cys-Glu-His-D-Phe-Arg-Trp-Cys]-NH₂ |
| 47 | Ac-D-Arg-cyclo[Cys-Glu-His-Phe-Arg-Trp-Cys]-NH₂ |
| 48 | Ac-Arg-cyclo[Cys-Glu-His-D-Phe-Arg-Trp-Cys]-NH₂ |
| 49 | Ac-Arg-cyclo[Cys-Glu-His-D-Phe-Arg-Trp-Cys]-OH |
| 50 | Ac-Arg-cyclo[Cys-Glu-His-(4-Cl-D-Phe)-Arg-Trp-Cys]-NH₂ |
| 51 | Ac-Arg-cyclo[Cys-Glu-(1-Me-His)-D-Phe-Arg-Trp-Cys]-NH₂ |
| 52 | Ac-D-Arg-cyclo[Cys-Glu-His-D-Phe-Arg-Trp-Cys]-NH₂ |
| 53 | Ac-D-Arg-cyclo[Cys-Glu-His-D-Phe-Arg-Trp-Cys]-OH |
| 54 | Ac-hArg-cyclo[Cys-Glu-His-D-Phe-Arg-Trp-Cys]-NH₂ |
| 55 | Ac-Cit-cyclo[Cys-Glu-His-D-Phe-Arg-Trp-Cys]-NH₂ |
| 56 | Ac-Cit-cyclo[Cys-Glu-(1-Me-His)-D-Phe-Arg-Trp-Cys]-NH₂ |
| 57 | Ac-Leu-cyclo[Cys-Glu-His-D-Phe-Arg-Trp-Cys]-NH₂ |
| 58 | Ac-Lys-cyclo[Cys-Glu-His-D-Phe-Arg-Trp-Cys]-NH₂ |
| 59 | Ac-Lys(ipr)-cyclo[Cys-Glu-His-D-Phe-Arg-Trp-Cys]-NH₂ |
| 60 | Ac-nLeu-cyclo[Cys-Glu-His-D-Phe-Arg-Trp-Cys]-NH₂ |
| 61 | Ac-nLeu-cyclo[Cys-Glu-His-D-Phe-Arg-Trp-Cys]-Ser-Pro-NH₂ |
| 62 | Ac-Orn-cyclo[Cys-Glu-His-D-Phe-Arg-Trp-Cys]-NH₂ |
| 63 | Ac-Val-cyclo[Cys-Glu-His-D-Phe-Arg-Trp-Cys]-NH₂ |
| 64 | N-(2-naphthalenesulfonyl)-D-Arg-cyclo[Cys-Glu-His-D-Phe-Arg-Trp-Cys]-NH₂ |
| 65 | N-(2-naphthalenesulfonylamino-4-oxo-butyryl)-D-Arg-cyclo[Cys-Glu-His-D-Phe-Arg-Trp-Cys]-NH₂ |
| 66 | 3-(4-hydroxyphenyl)propionyl-Arg-cyclo[Cys-Glu-His-D-Phe-Arg-Trp-Cys]-NH₂ |
| 67 | 3-(4-methylbenzoyl)propionyl-Arg-cyclo[Cys-Glu-His-D-Phe-Arg-Trp-Cys]-NH₂ |
| 68 | Tyr-Arg-cyclo[Cys-Glu-His-D-Phe-Arg-Trp-Cys]-NH₂ |
| 69 | Tyr-Arg-cyclo[Cys-Glu-His-D-Phe-Arg-Trp-Cys]-OH |
| 70 | Tyr-Arg-cyclo[Cys-Glu-His-D-Phe-Arg-Trp-Cys]-NH—(CH₂)₆—NH₂ |
| 71 | Tyr-Arg-cyclo[Cys-Glu-His-D-Phe-Arg-Trp-Cys]-Glu-NH₂ |
| 72 | Ac-Tyr-Arg-cyclo[Cys-Glu-His-D-Phe-Arg-Trp-Cys]-NH₂ |
| 73 | Ac-Tyr-Arg-cyclo[Cys-Glu-His-D-Phe-Arg-Trp-Cys]-OH |
| 74 | N-succinyl-Tyr-Arg-cyclo[Cys-Glu-His-D-Phe-Arg-Trp-Cys]-NH₂ |
| 75 | N-glutaryl-Tyr-Arg-cyclo[Cys-Glu-His-D-Phe-Arg-Trp-Cys]-NH₂ |
| 76 | N-glutaryl-Tyr-Arg-cyclo[Cys-Glu-His-D-Phe-Arg-Trp-Cys]-OH |
| 77 | gluconoyl-Tyr-Arg-cyclo[Cys-Glu-His-D-Phe-Arg-Trp-Cys]-NH₂ |
| 78 | Ac-Tyr-Arg-cyclo[Cys-Glu-His-D-Phe-Arg-Trp-Cys] alcohol |
| 79 | Ac-Tyr-D-Arg-cyclo[Cys-Glu-His-D-Phe-Arg-Trp-Cys]-NH₂ |
| 80 | Ac-Tyr-Arg-cyclo[D-Cys-Glu-His-D-Phe-Arg-Trp-Cys]-NH₂ |
| 81 | Ac-Tyr-Arg-cyclo[Cys-Glu-(1-Me-His)-D-Phe-Arg-Trp-Cys]-NH₂ |
| 82 | Ac-Tyr-Arg-cyclo[Cys-Glu-(1-Me-D-His)-D-Phe-Arg-Trp-Cys]-NH₂ |
| 83 | Ac-Tyr-Arg-cyclo[Cys-Glu-His-(4-F-D-Phe)-Arg-Trp-Cys]-NH₂ |
| 84 | Ac-Tyr-Arg-cyclo[Cys-Glu-(1-Me-His)-(4-F-D-Phe)-Arg-Trp-Cys]-NH₂ |
| 85 | Ac-Tyr-Arg-cyclo[Cys-Glu-(1-Me-D-His)-(4-F-D-Phe)-Arg-Trp-Cys]-NH₂ |
| 86 | Ac-Tyr-Arg-cyclo[Cys-Glu-His-(4-Cl-D-Phe)-Arg-Trp-Cys]-NH₂ |
| 87 | Ac-Arg-cyclo[Cys-Glu-(1-Me-His)-(4-Cl-D-Phe)-Arg-Trp-Cys]-NH₂ |
| 88 | Ac-Tyr-Arg-cyclo[Cys-Glu-(1-Me-D-His)-(4-Cl-D-Phe)-Arg-Trp-Cys]-NH₂ |
| 89 | Ac-Tyr-Arg-cyclo[Cys-Glu-His-(4-Br-D-Phe)-Arg-Trp-Cys]-NH₂ |
| 90 | Ac-Tyr-Arg-cyclo[Cys-Glu-(1-Me-His)-(4-Br-D-Phe)-Arg-Trp-Cys]-NH₂ |
| 91 | Ac-Tyr-Arg-cyclo[Cys-Glu-(1-Me-D-His)-(4-Br-D-Phe)-Arg-Trp-Cys]-NH₂ |
| 92 | Ac-Tyr-Arg-cyclo[Cys-Glu-His-(4-Me-D-Phe)-Arg-Trp-Cys]-NH₂ |
| 93 | Ac-Tyr-Arg-cyclo[Cys-Glu-His-(4-OMe-D-Phe)-Arg-Trp-Cys]-NH₂ |
| 94 | Ac-Tyr-Arg-cyclo[Cys-Glu-(1-Me-His)-(4-OMe-D-Phe)-Arg-Trp-Cys]-NH₂ |
| 95 | Ac-Tyr-Arg-cyclo[Cys-Glu-(1-Me-D-His)-(4-OMe-D-Phe)-Arg-Trp-Cys]-NH₂ |
| 96 | Ac-Tyr-Arg-cyclo[Cys-Glu-(3-Me-His)-D-Phe-Arg-Trp-Cys]-NH₂ |
| 97 | Ac-Tyr-Arg-cyclo[Cys-Glu-(5-Me-His)-D-Phe-Arg-Trp-Cys]-NH₂ |
| 98 | Ac-Tyr-Arg-cyclo[Cys-Glu-(5-Me-D-His)-D-Phe-Arg-Trp-Cys]-NH₂ |
| 99 | Ac-Tyr-Arg-cyclo[Cys-Glu-(1-benzyl-His)-D-Phe-Arg-Trp-Cys]-NH₂ |
| 100 | Ac-Tyr-Arg-cyclo[Cys-Glu-(1-benzyl-D-His)-D-Phe-Arg-Trp-Cys]-NH₂ |
| 101 | Ac-Tyr-Arg-cyclo[Cys-Glu-(1-Bom-His)-D-Phe-Arg-Trp-Cys]-NH₂ |
| 102 | Ac-Tyr-Arg-cyclo[Cys-Glu-(1-pyrazolyl-Ala)-D-Phe-Arg-Trp-Cys]-NH₂ |
| 103 | Ac-Tyr-Arg-cyclo[Cys-Glu-(4-phenyl-1H-imidazol-2-yl-Ala)-D-Phe-Arg-Trp-Cys]-NH₂ |
| 104 | Ac-Tyr-Arg-cyclo[Cys-Glu-(4-phenyl-1H-imidazol-2-yl-D-Ala)-D-Phe-Arg-Trp-Cys]-NH₂ |
| 105 | Ac-Tyr-Arg-cyclo[Cys-Glu-(2-pyrazine-Ala)-D-Phe-Arg-Trp-Cys]-NH₂ |
| 106 | Ac-Tyr-Arg-cyclo[Cys-Glu-(β-(1,2,4-triazol-3-yl))-Ala)-D-Phe-Arg-Trp-Cys]-NH₂ |
| 107 | Ac-Tyr-Arg-cyclo[Cys-Glu-(β-(1,2,4-triazol-3-yl))-D-Ala)-D-Phe-Arg-Trp-Cys]-NH₂ |
| 108 | Ac-Tyr-Arg-cyclo[Cys-Glu-(β-((1-benzyl)-1,2,4-triazol-3-yl))-D-Ala)-D-Phe-Arg-Trp-Cys]-NH₂ |
| 109 | Ac-Tyr-Arg-cyclo[Cys-Glu-(β-((1-benzyl)-1,2,4-triazol-3-yl))-D-Ala)-D-Phe-Arg-Trp-Cys]-NH₂ |
| 110 | Ac-Tyr-Arg-cyclo[Cys-Glu-(β-(2-furyl)-Ala)-D-Phe-Arg-Trp-Cys]-NH₂ |
| 111 | Ac-Tyr-Arg-cyclo[Cys-Glu-(β-(thien-2-yl)-Ala)-D-Phe-Arg-Trp-Cys]-NH₂ |
| 112 | Ac-Tyr-Arg-cyclo[Cys-Glu-(β-(1,3-thiazol-4-yl)-Ala)-D-Phe-Arg-Trp-Cys]-NH₂ |
| 113 | Ac-Tyr-Arg-cyclo[Cys-Glu-(β-(pyridin-4-yl)-Ala)-D-Phe-Arg-Trp-Cys]-NH₂ |
| 114 | Ac-Tyr-Arg-cyclo[Cys-Glu-His-D-Phe-Arg-Trp-Cys]-glycinol |
| 115 | Ac-Tyr-Arg-cyclo[Cys-Glu-His-D-Phe-Arg-Trp-Cys]-2-(2-aminoethoxy)ethanol |
| 116 | Ac-Tyr-Arg-cyclo[Cys-Glu-His-D-Phe-Arg-Trp-Cys]-Ser alcohol |
| 117 | Ac-Tyr-Arg-cyclo[Cys-Glu-His-D-Phe-Arg-Trp-Cys]-NH-(CH₂)₆-NH₂ |
| 118 | Ac-Tyr-Arg-cyclo[Cys-Glu-His-D-Phe-Arg-Trp-Cys]-Glu-NH₂ |
| 119 | Ac-Tyr-Arg-cyclo[Cys-Glu-His-D-Phe-Arg-Trp-Cys]-Ser-Pro-NH₂ |
| 120 | Ac-Tyr-Arg-cyclo[Cys-Glu-His-D-Phe-Arg-Trp-Cys]-Ser-Pro alcohol |
| 121 | Ac-Tyr-Arg-cyclo[Cys-Glu-His-D-Phe-Arg-Trp-Cys]-Lys-Pro-NH₂ |
| 122 | Ac-Tyr-Arg-cyclo[Cys-Glu-His-D-Phe-Arg-Trp-Cys]-Lys-Pro alcohol |
| 123 | Ac-Tyr-Arg-cyclo[Cys-Glu-His-D-Phe-Arg-Trp-Cys]-Arg-Phe-NH₂ |
| 124 | Ac-Tyr-Cit-cyclo[Cys-Glu-His-D-Phe-Arg-Trp-Cys]-NH₂ |
| 125 | Ac-Tyr-Cit-cyclo[Cys-Glu-(1-Me-His)-D-Phe-Arg-Trp-Cys]-NH₂ |
| 126 | Ac-Tyr-hArg-cyclo[Cys-Glu-His-D-Phe-Arg-Trp-Cys]-NH₂ |
| 127 | Ac-Tyr-(1-β-hArg)-cyclo[Cys-Glu-His-D-Phe-Arg-Trp-Cys]-NH₂ |
| 128 | Ac-Tyr-Lys-cyclo[Cys-Glu-His-D-Phe-Arg-Trp-Cys]-NH₂ |
| 129 | Ac-Tyr-Ser-cyclo[Cys-Glu-His-D-Phe-Arg-Trp-Cys]-NH₂ |
| 130 | Ac-Tyr-Val-cyclo[Cys-Glu-His-D-Phe-Arg-Trp-Cys]-NH₂ |
| 131 | N-succinyl-Tyr-Arg-cyclo[Cys-Glu-His-D-Phe-Arg-Trp-Cys]-OH |
| 132 | cyclo[hCys-His-D-Phe-Arg-Trp-Cys]-NH₂ |
| 133 | cyclo[hCys-His-D-Phe-Arg-Trp-Cys]-OH |
| 134 | cyclo[hCys-His-(4-F-D-Phe)-Arg-Trp-Cys]-NH₂ |
| 135 | cyclo[hCys-His-(4-Cl-D-Phe)-Arg-Trp-Cys]-NH₂ |
| 136 | Ac-cyclo[hCys-His-Phe-Arg-Trp-Cys]-NH₂ |
| 137 | Ac-cyclo[hCys-His-D-Phe-Arg-Trp-Cys]-NH₂ |
| 138 | Ac-cyclo[hCys-His-D-Phe-Arg-Trp-Cys]-OH |
| 139 | Ac-cyclo[hCys-His-(4-F-D-Phe)-Arg-Trp-Cys]-NH₂ |
| 140 | Ac-cyclo[hCys-His-(4-Cl-D-Phe)-Arg-Trp-Cys]-NH₂ |
| 141 | N-cyclopropanecarbonyl-cyclo[hCys-His-D-Phe-Arg-Trp-Cys]-NH₂ |
| 142 | N-cyclobutanecarbonyl-cyclo[hCys-His-D-Phe-Arg-Trp-Cys]-NH₂ |
| 143 | N-cyclopentanecarbonyl-cyclo[hCys-His-D-Phe-Arg-Trp-Cys]-NH₂ |
| 144 | N-cyclohexanecarbonyl-cyclo[hCys-His-D-Phe-Arg-Trp-Cys]-NH₂ |
| 145 | N-hexanoyl-cyclo[hCys-His-D-Phe-Arg-Trp-Cys]-NH₂ |
| 146 | N-benzoyl-cyclo[hCys-His-D-Phe-Arg-Trp-Cys]-NH₂ |
| 147 | 4-phenylbutyryl-cyclo[hCys-His-D-Phe-Arg-Trp-Cys]-NH₂ |
| 148 | 3-guanidinopropionyl-cyclo[hCys-His-D-Phe-Arg-Trp-Cys]-NH₂ |
| 149 | 5-guanidinovaleryl-cyclo[hCys-His-D-Phe-Arg-Trp-Cys]-NH₂ |
| 150 | N-phenylsulfonyl-cyclo[hCys-His-D-Phe-Arg-Trp-Cys]-NH₂ |
| 151 | N-(2-naphthalenesulfonyl)-cyclo[hCys-His-D-Phe-Arg-Trp-Cys]-NH₂ |
| 152 | N-(4-phenylsulfonamido-4-oxo-butyryl)-cyclo[hCys-His-D-Phe-Arg-Trp-Cys]-NH₂ |
| 153 | Arg-cyclo[hCys-His-D-Phe-Arg-Trp-Cys]-NH₂ |
| 154 | D-Arg-cyclo[hCys-His-D-Phe-Arg-Trp-Cys]-NH₂ |
| 155 | Arg-cyclo[hCys-His-D-Phe-Arg-Trp-Cys]-OH |
| 156 | Arg-cyclo[hCys-(1-Me-His)-D-Phe-Arg-Trp-Cys]-NH₂ |
| 157 | Arg-cyclo[hCys-(1-Me-D-His)-D-Phe-Arg-Trp-Cys]-NH₂ |
| 158 | Ac-Arg-cyclo[hCys-His-D-Phe-Arg-Trp-Cys]-NH₂ |
| 159 | Ac-Arg-cyclo[hCys-His-D-Phe-Arg-Trp-Cys]-OH |
| 160 | Ac-nLeu-cyclo[hCys-His-D-Phe-Arg-Trp-Cys]-NH₂ |
| 161 | phenylsulfonyl-Gly-cyclo[hCys-His-D-Phe-Arg-Trp-Cys]-NH₂ |
| 162 | Tyr-Arg-cyclo[hCys-His-D-Phe-Arg-Trp-Cys]-NH₂ |
| 163 | Tyr-Arg-cyclo[hCys-His-D-Phe-Arg-Trp-Cys]-OH |
| 164 | Ac-Tyr-Arg-cyclo[hCys-His-D-Phe-Arg-Trp-Cys]-NH₂ |
| 165 | Ac-Tyr-Arg-cyclo[hCys-His-D-Phe-Arg-Trp-Cys]-OH |
| 166 | Ac-Tyr-Arg-cyclo[hCys-Glu-His-D-Phe-Arg-Trp-Cys]-NH₂ |
| 167 | Ac-cyclo[hCys-His-(β-cyclohexyl-D-Ala)-Arg-Trp-Cys]-NH₂ |
| 168 | Ac-cyclo[hCys-His-D-Phe-Arg-Trp-penicillamine]-NH₂ |
| 169 | Ac-cyclo[hCys-His-(4-Cl-D-Phe)-Arg-Trp-penicillamine]-NH₂ |
| 170 | N-hexanoyl-cyclo[hCys-His-D-Phe-Arg-Trp-penicillamine]-NH₂ |
| 171 | N-cyclopentanecarbonyl-cyclo[hCys-His-D-Phe-Arg-Trp-penicillamine]-NH₂ |
| 172 | N-cyclohexanecarbonyl-cyclo[hCys-His-D-Phe-Arg-Trp-penicillamine]-NH₂ |
| 173 | N-benzoyl-cyclo[hCys-His-D-Phe-Arg-Trp-penicillamine]-NH₂ |
| 174 | 4-phenylbutyryl-cyclo[hCys-His-D-Phe-Arg-Trp-penicillamine]-NH₂ |
| 175 | N-phenylsulfonyl-cyclo[hCys-His-D-Phe-Arg-Trp-penicillamine]-NH₂ |
| 176 | (4-benzenesulfonamide)butyryl-cyclo[hCys-His-D-Phe-Arg-Trp-penicillamine]-NH₂ |
| 177 | Ac-nLeu-cyclo[hCys-His-D-Phe-Arg-Trp-penicillamine]-NH₂ |
| 178 | N-phenylsulfonyl-Gly-cyclo[hCys-His-D-Phe-Arg-Trp-penicillamine]-NH₂ |
| 179 | cyclo[3-thiopropionyl-His-D-Phe-Arg-Trp-hCys]-NH₂ |
| 180 | cyclo[Cys-His-D-Phe-Arg-Trp-hCys]-NH₂ |
| 181 | cyclo[Cys-His-(4-F-D-Phe)-Arg-Trp-hCys]-NH₂ |
| 182 | cyclo[Cys-His-(4-Cl-D-Phe)-Arg-Trp-hCys]-NH₂ |
| 183 | Ac-cyclo[Cys-His-D-Phe-Arg-Trp-hCys]-NH₂ |
| 184 | Ac-cyclo[Cys-His-(4-F-D-Phe)-Arg-Trp-hCys]-NH₂ |
| 185 | Ac-cyclo[Cys-His-(4-Cl-D-Phe)-Arg-Trp-hCys]-NH₂ |
| 186 | Arg-cyclo[Cys-His-D-Phe-Arg-Trp-hCys]-NH₂ |
| 187 | Arg-cyclo[Cys-His-(4-F-D-Phe)-Arg-Trp-hCys]-NH₂ |
| 188 | Arg-cyclo[Cys-His-(4-Cl-D-Phe)-Arg-Trp-hCys]-NH₂ |
| 189 | Ac-Arg-cyclo[Cys-His-D-Phe-Arg-Trp-hCys]-NH₂ |
| 190 | Ac-Arg-cyclo[Cys-His-(4-F-D-Phe)-Arg-Trp-hCys]-NH₂ |
| 191 | Ac-Arg-cyclo[Cys-His-(4-Cl-D-Phe)-Arg-Trp-hCys]-NH₂ |
| 192 | Ac-Tyr-Arg-cyclo[Cys-Glu-His-D-Phe-Arg-Trp-hCys]-NH₂ |
| 193 | Ac-cyclo[hCys-His-D-Phe-Arg-Trp-hCys]-NH₂ |
| 194 | Arg-cyclo[hCys-His-D-Phe-Arg-Trp-hCys]-NH₂ |
| 195 | Ac-Arg-cyclo[hCys-His-D-Phe-Arg-Trp-hCys]-NH₂ |
| 196 | Ac-Tyr-Arg-cyclo[hCys-His-D-Phe-Arg-Trp-hCys]-NH₂ |
| 197 | Ac-Tyr-Arg-cyclo[hCys-Glu-His-D-Phe-Arg-Trp-hCys]-NH₂ |
| 198 | Ac-cyclo(S—CH₂—S)[Cys-His-D-Phe-Arg-Trp-Cys]-NH₂ |

Many of those peptides identified as MC4R agonist peptides have been found to be MC3R agonists. In particular, an “MC3R agonist peptide” for use in the present invention includes peptides of Structural Formula II (SEQ ID NO:200):

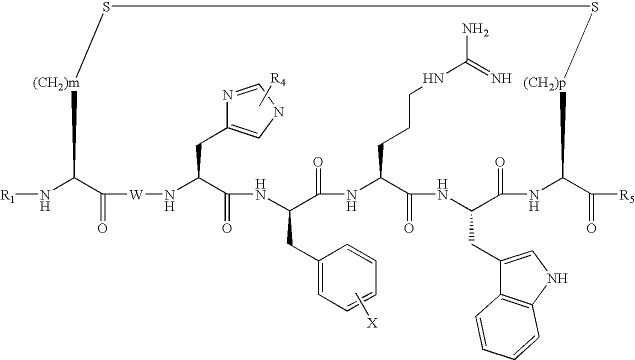

and pharmaceutically acceptable salts thereof, wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac—Tyr-β-hArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Tyr-Arg-, N-succinyl-Tyr-Arg-, R⁶—SO₂NHC(O)CH₂CH₂C(O)—, R⁶—SO₂NHC(O)CH₂CH₂C(O)Arg—, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH₂)_{q}C(O)—, 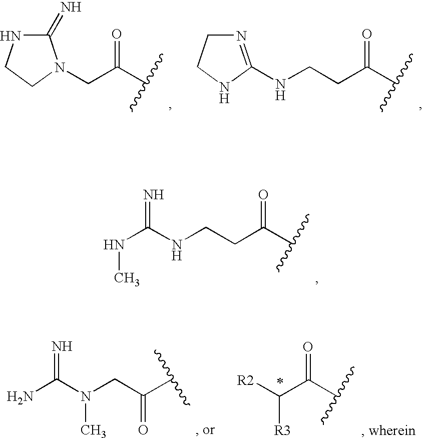
R² is —H, —NH₂, —NHC(O)CH₃, —NHC(O)(CH₂)₁₋₄CH₃, —NH-TyrC(O)CH₃, R⁶SO₂NH—, Ac-Cya-NH—, Tyr-NH—, HO—(C₆H₅)—CH₂CH₂C(O)NH—, or CH₃—(C₆H₅)—C(O)CH₂CH₂C(O)NH—;
R³ is C₁-C₄ straight or branched alkyl, NH₂—CH₂—(CH₂)_{q}—, HO—CH₂—, (CH₃)₂CHNH(CH₂)₄—, R⁶(CH₂)_{q}—, R⁶SO₂NH—, Ser, Ile, 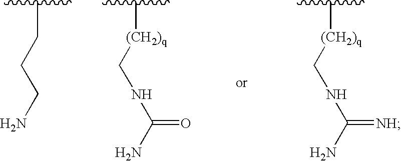
q is 0, 1, 2, or 3;
R⁶ is a phenyl or C₈-C₁₄ bicyclic aryl;
m is 1 or 2;
p is 1 or 2;
R⁴ is H, C₁-C₄ straight or branched alkyl, phenyl, benzyl, or (C₆H₅)—CH₂—O—CH₂—;
X is H, Cl, F, Br, methyl, or methoxy; and
R⁵ is —NH₂, —OH, glycinol, NH₂-Pro-Ser-, NH₂-Pro-Lys, HO-Ser-, HO-Pro-Ser-, HO-Lys-, -Ser alcohol, -Ser-Pro alcohol, -Lys-Pro alcohol, HOCH₂CH₂—O—CH₂CH₂NH—, NH₂-Phe-Arg-, NH₂-Glu-, NH₂CH₂RCH₂NH—, RHN—, or RO— where R is a C₁-C₄ straight or branched alkyl,

with the proviso that compounds having the following combinations are excluded:
a) W is absent, R¹=Ac, m=1, p=1, R⁴=H, X=H, and R⁵=NH₂;
b) W=Glu, R¹=H, m=1, p=1, R⁴=H, X=H, and R⁵=NH₂;
c) W=Glu, R¹=Ac, m=1, p=1, R⁴=1-methyl-, X=H, and R⁵=NH₂;
d) W=Glu, R¹=Arg-, m=1, p=1, R⁴=H, X=H, and R⁵=OH;
e) W=Glu, R¹=Ac-D-Arg-, m=1, p=1, R⁴=H, X=H, and R⁵=NH₂;
f) W=Glu, R¹=Ac-Arg-, m=1, p=1, R⁴=H, X=H, and R=OH;
g) W=Glu, R¹=Ac-D-Arg-, m=1, p=1, R⁴=H, X=H, and R⁵=OH;
h) W=Glu, R¹=Ac-Cit, m=1, p=1, R⁴=1-methyl-, X=H, and R⁵=NH₂;
i) W=Glu, R¹=N-glutaryl-Tyr-Arg, m=1, p=1, R⁴=H, X=H, and R⁵=OH;
j) W=Glu, R¹=R²—CH(R³)—C(O)—, R²=—NH-TyrC(O)CH₃, R³=(CH₂)_{q}—CH₂—NH—C(O)—NH₂, q=2, m=1, p=1, R⁴=1-methyl-, X=H,and R=NH₂;
k) W=Glu, R¹=N-succinyl-Tyr-Arg-, m=1, p=1, R⁴=H, X=H, and R⁵=OH;
l) W=Glu, R¹=R²—CH(R³)—C(O)—, R²=—NH-TyrC(O)CH₃, R³=(CH₂)_{q}—CH₂—NH—C(NH)—NH₂, q=2, m=2, p=1, R⁴=H, X=H, and R⁵=NH₂;
m) W is absent, R¹=H, m=1, p=2, R⁴=H, X=H, and R⁵=NH₂;
n) W is absent, R¹=H, m=1, p=2, R⁴=H, X=4-fluoro-, and R⁵=NH₂; and
o) W=Glu, R¹=R²—CH(R³)—C(O)—, R²=—NH-TyrC(O)CH₃, R³=(CH₂)_{q}—CH₂—NH—C(NH)—NH₂, q=2, m=1, p=2, R⁴=H, X=H, and R⁵=NH₂.

Another group of MC3R agonist peptides for use in the present invention include compounds from a subgenus of Structural Formula I (supra). This subgenus, shown here as Structural Formula III (SEQ ID NO:201), includes the following:

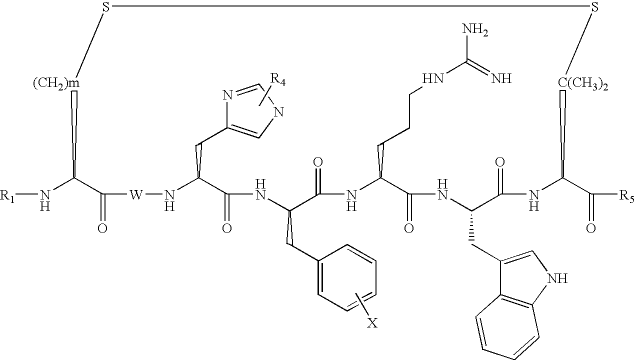

and pharmaceutically acceptable salts thereof, wherein
W is Glu, Gln, Asp, Asn, Ala, Gly, Thr, Ser, Pro, Met, Ile, Val, Arg, His, Tyr, Trp, Phe, Lys, Leu, Cya, or is absent;
R¹ is —H, —C(O)CH₃, —C(O)(CH₂)₁₋₄CH₃, —C(O)(CH₂)₁₋₄NHC(NH)NH₂, Tyr-βArg-, Ac-Tyr-βhArg-, gluconoyl-Tyr-Arg-, Ac-diaminobutyryl-, Ac-diaminopropionyl-, N-propionyl-, N-butyryl-, N-valeryl-, N-methyl-Tyr-Arg-, N-glutaryl-Try-Arg-, N-succinyl-Tyr-Arg-, R⁶—SO₂NHC(O)CH₂CH₂C(O)—, R⁶—SO₂NHC(O)CH₂CH₂C(O)Arg-, R⁶—SO₂NHCH₂CH₂CH₂C(O)—, C₃-C₇ cycloalkylcarbonyl, phenylsulfonyl, C₈-C₁₄ bicyclic arylsulfonyl, phenyl-(CH₂)_{q}C(O)—, C₈-C₁₄ bicyclic aryl-(CH2)_{q}C(O)—, 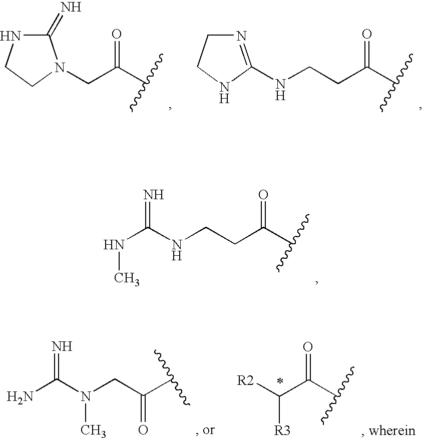
R² is —H, —NH₂, —NHC(O)CH₃, —NHC(O)(CH₂)₁₋₄CH₃, —NH-TyrC(O)CH₃, R⁶SO₂NH-, Ac-Cya-NH-, Tyr-NH-, HO—(C₆H₅)—CH₂CH₂C(O)NH—, or CH₃—(C₆H₅)—C(O)CH₂CH₂C(O)NH—;
R³ is C¹-C₄ straight or branched alkyl, NH₂—CH₂—(CH₂)_{q}—, HO—CH₂—, (CH₃)₂CHNH(CH₂)₄—, R⁶(CH²)_{q—}, R⁶SO₂NH—, Ser, Ile, 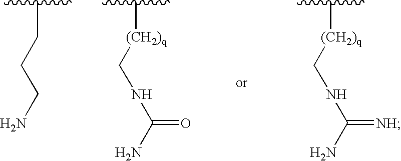
q is 0, 1, 2, or 3;
R⁶ is a phenyl or C₈-C₁₄ bicyclic aryl;
m is 1 or 2;
p is 1 or 2;
R⁴ is H, C₁-C₄ straight or branched alkyl, phenyl, benzyl, or (C₆H₅)—CH₂—O—CH₂—;
X is H, Cl, F, Br, methyl, or methoxy; and
R⁵ is —NH₂, —OH, glycinol, NH₂-Pro-Ser-, NH₂-Pro-Lys, HO-Ser-, HO-Pro-Ser-, HO-Lys-, -Ser alcohol, -Ser-Pro alcohol, -Lys-Pro alcohol, HOCH₂CH₂—O—CH₂CH₂NH—, NH₂-Phe-Arg-, NH₂-Glu-, NH₂CH₂RCH₂NH—, RHN—, or RO— where R is a C₁-C₄ straight or branched alkyl.

Another group of MC3R agonist peptides for use in the present invention includes compounds listed in Table 1 (“Specific MC4R agonist peptides”) with the proviso that peptides having Compound Nos. 1, 29, 34, 45, 47, 49, 53, 56, 76, 78, 82, 85, 88, 95, 100, 102, 104, 105, 107, 110, 111, 112, 125, 131, 136, 166, 167, 179, 180, 181, 192, and 198 are excluded.

A preferred group of MC3R agonist peptides for use in the present invention includes peptides having Compound Nos. 50, 86, 89, 92, 121, 122, 134, 135, 137, 141 through 149, 153, 158, 160, 161, 162, 164, 169 through 174, 177, and 191. A more preferred group of MC3R agonist peptides for use in the present invention includes peptides having Compound Nos. 135, 143, 147, 149, 160, 161, 169, and 177. Most preferred MC3R agonists for use in the present invention include Compound No. 149, denoted by the name 5-guanidinovaleryl-cyclo[hCys-HisD-Phe-Arg-Trp-Cys]-NH₂, and Compound No. 160, denoted by the name Ac-nLeu-cyclo[hCys-His-D-Phe-Arg-Trp-Cys]- NH₂.

As used herein, “C₁-C₄ straight or branched alkyl” means a straight chained or branched hydrocarbon having 1 to 4 carbon atoms, which is completely saturated and unsubstituted. “C₃-C₇ cycloalkyl” refers to a saturated, unsubstituted hydrocarbon ring having 3 to 7 carbon atoms. A “C₁-C₄ straight or branched heteroalkyl” refers to a straight chained or branched hydrocarbon having 1 to 4 carbon atoms, which is completely saturated and unsubstituted, that also contains at least one “heteroatom.” A “heteroatom” is nitrogen, oxygen, or sulfur. “C₃-C₇ heterocycloalkyl” refers to a saturated, unsubstituted hydrocarbon ring having 3 to 7 carbon atoms, which also contains at least one “heteroatom.” C₁-C₄ straight or branched alkyl, C₃-C₇ cycloalkyl, C₁-C₄ straight or branched heteroalkyl, and C₃-C₇ heterocycloalkyl may be used as generic modifiers to describe a genus of substituents on another functional group such as a carbonyl, sulfonyl, or sulfonamide. For example, a “C₃-C₇ cycloalkylcarbonyl” refers to a genus of saturated, unsubstituted hydrocarbon rings having 3 to 7 carbon atoms that are bonded to a carbonyl group.

A “C₈-C₁₄ bicyclic aryl” refers to two or three hydrocarbon rings fused together, having 8 to 14 carbon atoms, such as naphthalene. A C₈-C₁₄ bicyclic aryl ring system has at least one aromatic ring. A “5- or 6-membered heteroaryl” refers to a monocyclic aromatic ring having 5 or 6 atoms, of which 1-4 atoms are heteroatoms. An “8- to 14-membered bicyclic heteroaryl” ring refers to two or three hydrocarbon rings fused together, having 8 to 14 atoms, at least one aromatic ring, and 1-4 heteroatoms.

A phenyl, benzyl, benzoyl, C₈-C₁₄ bicyclic aryl, 5- or 6-membered heteroaryl, or 8- to 14-membered bicyclic heteroaryl may be unsubstituted or substituted with C₁-C₄ straight or branched alkyl, F, Cl, Br, —OH, methoxy, phenyl, benzyl, benzoyl, or benzyloxymethyl. Furthermore, phenyl, benzyl, benzoyl, C₈-C₁₄ bicyclic aryl, 5- or 6-membered heteroaryl, and 8- to 14-membered bicyclic heteroaryl may be used as generic modifiers to describe a genus of substituents on another functional group such as a carbonyl, sulfonyl, or sulfonamide. For example, a “C₈-C₁₄ bicyclic arylsulfonyl” refers to a genus of bicyclic aryl rings having 8 to 14 carbon atoms that are bonded to a sulfonyl group.

Modified amino acids are indicated by parentheses around the amino acid and the modification thereto (e.g., (4—Cl-D-Phe) is a 4-chloro modification on the D-isomer of phenylalanine). With respect to moieties depicted in Structural Formula I, Structural Formula II, and Structural Formula III, the single letter designations are as defined and do not refer to single letter amino acids corresponding to those letters.

The letter “D” preceding the above-mentioned 3-letter abbreviations, e.g., “D-Phe,” means the D-form of the amino acid. When the single letter abbreviation is used for an amino acid, a “d” will precede the letter to designate the D-form of the amino acid (e.g., dF =D-Phe).

An “amino alcohol” is an amino acid that has been modified by reducing the carbonyl group of the C-terminus to a methylene group. Amino alcohols are denoted by the general nomenclature “Xaa alcohol,” wherein Xaa is the specific amino acid from which the carbonyl group has been removed. To illustrate, “Ser alcohol” has the structure H₂N—CH(CH₂OH)—CH₂OH as opposed to the Ser amino acid structure of H₂N—CH(CH₂OH)—COOH.

“Single bond,” as used herein, refers to a structure that does not contain an amino acid at the specified position. It is used to signify that an amino acid is absent from that position such that the carbonyl adjacent to that position on one side and the amine adjacent to that position on the other side form a peptide bond with each other.

“*” means that both the D— and L— isomers are possible.

“Ac” refers to acetyl (i.e., —C(O)CH₃).

“Orn” refers to ornithine.

“hCys” refers to homocysteine.

“hArg” refers to homoarginine.

“Lys(ipr)” refers to lysine(N-isopropyl).

“Cit” refers to citrulline.

“nLeu” refers to norleucine.

“Me” refers to methyl.

“OMe” refers to methoxy.

“Cya” refers to cysteic acid.

“Dap” refers to diaminopropionyl.

“Dab” refers to diaminobutyryl.

“Pharmaceutically-acceptable salt” refers to salts of the compounds of the Structural Formula I, Structural Formula II, or Structural Formula III that are substantially non-toxic to mammals. Typical pharmaceutically acceptable salts include those salts prepared by reaction of the compounds of the present invention with a mineral or organic acid or an organic or inorganic base. Such salts are known as acid addition and base addition salts, respectively. It should be recognized that the particular counterion forming a part of any salt of this invention is not of a critical nature, so long as the salt as a whole is pharmaceutically acceptable and as long as the counterion does not contribute undesired qualities to the salt as a whole.

A pharmaceutical “acid addition salt” is a salt formed by reaction of the free base form of a compound of formula I with a pharmaceutical acid, such as described in the Encyclopedia of Pharmaceutical Technology, editors James Swarbrick and James C. Boylan, Vol. 13 (1996), “Preservation of Pharmaceutical Products to Salt Forms of Drugs and Absorption.” Specific salt forms include, but are not limited to the: acetate, benzoate, benzenesulfonate, 4-chlorobenzenesulfonate; citrate; ethanesulfonate; fumarate; d-gluconate; d-glucuronate; glutarate; glycolate; hippurate; hydrochloride; 2-hydroxyethanesulfonate; dl-lactate; maleate; d-malate; l-malate; malonate; d-mandelate; l-mandelate; methanesulfonate; 1,5-napthalenedisulfonate; 2-naphthalenesulfonate; phosphate; salicylate; succinate; sulfate; d-tartrate; l-tartrate; and p-toluenesulfonate.

A pharmaceutical “base addition salt” is a salt formed by reaction of the free acid form of a compound of formula I with a pharmaceutical base, such as described in the Encyclopedia of Pharmaceutical Technology, supra. Specific salt forms include, but are not limited to the: calcium, diethanolamine, diethylamine, ethylenediamine, lysine, magnesium, piperazine, potassium, sodium, and tromethamine (Tris, Trizma) salts.

The term “active ingredient” means the MC3R agonist peptides generically described by Structural Formula II and Structural Formula III, as well as the salts of such compounds.

The term “pharmaceutically acceptable” means that the carrier, diluent, excipients, and salt must be compatible with the other ingredients of the composition and not clinically deleterious to the recipient thereof. Pharmaceutical compositions of the present invention are prepared by procedures known in the art using well-known and readily available ingredients.

The term “agonist” includes any molecule that has affinity for the MC3 receptor, producing a measurable biological activity associated with weight loss in cells, tissues and organisms containing the MC3 receptor. In a similar manner, an “inverse agonist” includes any molecule that has affinity for the MC3 receptor, producing a decreased intrinsic activity of the cell containing the MC3 receptor and is associated with weight gain in cells, tissues, and organisms containing the MC3 or MC4 receptor. The term “antagonist” includes any molecule that partially or fully blocks, inhibits, or neutralizes a biological activity of the MC3 receptor. Assays measuring such activities are well known in the art.

The term “selective” means having an activation preference for a certain receptor over other receptors that can be quantified based on whole cell, tissue, or organism assays that demonstrate receptor activity. Selectivity is ascertained by comparison of EC₅₀ values at the relevant receptors referenced.

The term “weight loss” includes any decrease in the mass of a patient. Weight loss may include overall loss of mass by the patient or, alternatively, loss of fat mass by the patient.

The term “obesity,” also called corpulence or fatness, is the excessive accumulation of body fat, usually caused by the consumption of more calories than the body uses. The excess calories are then stored as fat, or adipose tissue. Overweight, if moderate, is not necessarily obesity, particularly in muscular or large-boned individuals. In general, however, a body weight twenty percent or more over the optimum tends to be associated with obesity.

A “metabolic disorder” of the present invention includes, but is not limited to, obesity, diabetes mellitus, cachexia, sarcopenia, frailty, and cardiovascular disorders. A “cardiovascular disorder” of the instant invention may include disorders such as dyslipidemias, atherosclerosis, elevated blood pressure, hypertension, stroke, hypercholesterolemia, and related pathological sequelae.

A “subject” or “patient” is a mammal, preferably a human. Nonetheless, other mammals may be subjects or patients, including companion animals such as dogs and cats, laboratory animals such as rats, mice, monkeys, and guinea pigs, and farm animals such as cows, sheep, pigs, and horses.

The term “a patient in need thereof” is a patient either suffering from the claimed pathological condition or sequela thereof or is a patient at a recognized risk thereof as determined by medical diagnosis, i.e., as determined by the attending physician.

The terms “treating,” “treatment,” and “therapy” as used herein refer to the management and care of a patient for the purpose of combating the disease, condition, or disorder. Treating includes the administration of an MC3R agonist peptide to prevent the onset of the symptoms or complications, alleviating the symptoms or complications, or eliminating the disease, condition, or disorder. Treating obesity may include the inhibition of food intake, the inhibition of weight gain, and/or inducing weight loss in patients in need thereof.

Treatment may include curative therapy, prophylactic therapy, and preventive therapy. An example of “preventive therapy” is the prevention or lessened targeted pathological condition or disorder. Those in need of treatment include those already with the disorder as well as those prone to have the disorder or those in whom the disorder is to be prevented.

“Pharmaceutically effective amount” means that amount of a compound, or salt thereof, that will elicit the biological or medical response of a tissue, system, or mammal and/or is capable of treating the conditions described herein, or that is capable of agonizing the MC3 and/or MC4 receptors. An “effective amount” of the peptide administered to a subject will also depend on the type and severity of the disease or condition and on the characteristics of the subject, such as general health, age, sex, body weight and tolerance to drugs. The recipient patient's physician should determine the therapeutic dose administered in light of the relevant circumstances.

A pharmaceutically effective amount can be administered prophylactically to a patient thought to be susceptible to development of a disease or condition. Such amount, when administered prophylactically to a patient, can also be effective to prevent or lessen the severity of the mediated condition. The dosage regimen utilizing the compounds of the present invention is selected by one of ordinary skill in the medical or veterinary arts, in view of a variety of factors, including, without limitation, the route of administration, the prior medical history of the recipient, the pathological condition or symptom being treated, the severity of the condition/symptom being treated, and the age and sex of the recipient patient. However, it will be understood that the therapeutic dose administered will be determined by the attending physician in the light of the relevant circumstances.

Generally, an effective minimum daily dose of a compound of the present invention will exceed about 0.01 mg. Typically, an effective maximum daily dose will not exceed about 1000 mg. More preferably, an effective minimum daily dose will be between about 0.05 mg and 50 mg, more preferably between 0.1 mg and 10 mg. Most preferably, an effective minimum daily dose of an MC3R agonist peptide in the present invention will exceed about 2 μg/kg and will not exceed about 20 μg/kg. The exact dose may be determined, in accordance with the standard practice in the medical arts of “dose titrating” the recipient; that is, initially administering a low dose of the compound, and gradually increasing the dose until the desired therapeutic effect is observed. The desired dose may be presented in a single dose or as divided doses administered at appropriate intervals.

Administration of an MC3R agonist peptide can be effected in a single daily dose, or the total daily dose may be administered in divided doses, two, three, or more times per day, or by continuous infusion. Where delivery is via transdermal forms, of course, administration is continuous.

Routes of administration of an MC3R agonist peptide include a variety of routes, including the oral, subcutaneous, topical, parenteral (e.g., intravenous and intramuscular), bronchial, or intranasal routes.

“Continuous infusion” of an MC3R agonist peptide refers to controlled parenteral delivery of the peptide to a patient for an extended period of time. Administration of the peptide may be accomplished by, but is not limited to, delivery via pump, depot, suppository, pessary, transdermal patch or other topical administration (such as buccal, sublingual, spray, ointment, creme, or gel) using, for example, subcutaneous, intramuscular, intraperitoneal, intravenous, intracerebral, or intraarterial administration.

A pump delivering the MC3R agonist peptide into the body may be implanted in the patient's body. Alternatively, the patient may wear a pump externally, being attached to the patient's body via catheter, needle, or some other connective means. Any pump that is suitable for the delivery of pharmaceuticals to a patient may be used. Examples include pumps such as those disclosed in U.S. Pat. No. 6,659,982.

A depot is a biocompatible polymer system containing the MC3R agonist peptide and delivering the peptide over time. Examples include microspheres, microcapsules, nanoparticles, liposomes, a hydrogel, or other polymeric implants. Preferred periods for delivery of agonist by depot include one week, two weeks, and one month periods. If needed, another depot will be delivered to the patient for continued delivery of peptide.

Engineering the MC3R agonist peptide to have a prolonged half-life will also result in continuous delivery of the MC3 receptor agonist to the receptor. Such modifications include conjugations with larger proteins such as albumin, antibody and antigen or chemical modifications that may increase half-life by linking fatty acids, polyethylene glycol (PEG) polymers, and other agents.

The MC3R agonist peptides may be used effectively alone or in combination with one or more additional active agents depending on the desired target therapy. Combination therapy includes administration of a single pharmaceutical dosage composition which contains a compound of Structural Formula I, Structural Formula II, or Structural Formula III, and one or more additional active agents, as well as administration of a compound of Structural Formula I, Structural Formula II, or Structural Formula III, and each active agent in its own separate pharmaceutical dosage formulation. Where separate dosage formulations are used, a compound of Structural Formula I, Structural Formula II, or Structural Formula III, and one or more additional active agents can be administered at essentially the same time, i.e., concurrently, or at separately staggered times, i.e., sequentially; combination therapy is understood to include all of these regimens.

A preferred combination therapy for the treatment of obesity is the use of an MC3R agonist peptide in combination with sibutramine (or active metabolites of sibutramine, e.g., desmethyl sibutramine and di-desmethyl sibutramine), preferably with sibutramine hydrochloride monohydrate. Another preferred combination is the use of an MC3R agonist peptide in combination with orlistat.

All peptides of the present invention can be synthesized by solid-phase synthesis methods (Merrifield, J. Am. Chem. Soc. 85:2149-54, 1963) either by manual or automated synthesis techniques. The automated assembly can be carried out using either as ABI 431A or 433A synthesizer.

The following examples are not intended to limit the invention in any way.

### EXAMPLE 1

### Construction of MC Receptor Expression Plasmids

Construction of human MC1 expression plasmid: Human MC1 cDNA is cloned by PCR using human genomic DNA (Clontech Cat. #6550-1) as a template. A forward hMC 1 gene-specific primer containing initiation codon (ATG) and EcoRI site and a reverse hMC1 gene specific primer containing a stop codon and XbaI site are used in the PCR. The full-length hMC1 cDNA generated by PCR is cloned into pUC18/SmaI plasmid (Pharmacia Cat. # 27-5266-01), and the correct hMC1 cDNA is confirmed by DNA sequencing. The sequenced pUC 18hMC1 is digested with EcoRI and XbaI, and the hMC1 cDNA fragment is then subcloned into pcDNA3.1 (Invitrogen Cat. # V790-20) to generate expression plasmid pCDNA3-hMC1.

Construction of human MC3 expression plasmid: Human MC3 cDNA is cloned by PCR using human genomic DNA (Clontech Cat. # 6550-1) as a template. A forward hMC3 gene-specific primer containing initiation codon (ATG) and EcoRI site and a reverse hMC3 gene specific primer containing a stop codon and XbaI site are used in the PCR. The full-length hMC3 cDNA generated by PCR is cloned into pUC18/SmaI plasmid (Pharmacia Cat# 27-5266-01), and the correct hMC3 cDNA is confirmed by DNA sequencing. The sequenced pUC 18hMC3 is digested with EcoRI and XbaI, and the hMC3 cDNA fragment is then subcloned into pcDNA3.1 (Invitrogen Cat. # V790-20) to generate expression plasmid pCDNA3-hMC3.

Construction of human MC4 expression plasmid: Human MC4 (hMC4) cDNA is cloned in a similar way as hMC3 cDNA by PCR using human fetal brain cDNA (Clontech Cat. # 7402-1) as a template. The hMC4 cDNA PCR product is digested with EcoRI/XbaI, and then subcloned into pCIneo (Promega Cat. # E1841) and sequenced. The resulting hMC4R plasmid has two mutations, which are then corrected to create the hMC4 cDNA encoding the correct hMC4 protein. The corrected hMC4 cDNA is then subcloned into pcDNA3.1 to generate expression plasmid pCDNA3-hMC4.

Construction of human MC5 expression plasmid: Human MC5 cDNA is cloned by PCR using human genomic DNA (Clontech Cat. # 6550-1) as a template. A forward hMC5 gene-specific primer containing initiation codon (ATG) and HindIII site and a reverse hMC5 gene specific primer containing a stop codon and XbaI site are used in the PCR. The full-length hMC5 cDNA generated by PCR is cloned into pUC18/SmaI plasmid (Pharmacia Cat. # 27-5266-01), and the correct hMC5 cDNA is confirmed by DNA sequencing. The sequenced pUC18hMC5 is digested with EcoRI and XbaI, and the hMC5 cDNA fragment is then subcloned into pcDNA3.1 (Invitrogen Cat. # V790-20) to generate expression plasmid pCDNA3-hMC5.

Stable HEK-293 cells expressing human MCRs: Stable 293 cells expressing all hMCRs are generated by co-transfecting HEK-293 cells with pCDNA3-hMC4R and a CRE-luciferase reporter plasmid following the protocol of Lipofectamine Plus Reagent (Invitrogen, Cat. # 10964-013). For selection of stable transfectants, the Genticin (G418) is added to the media at a concentration of 300 μg/mL 48 hours after the start of transfection. After 2-3 weeks, 40-50 of isolated clones are selected, propagated, and assayed for luciferase activity using a Luciferase Reporter Gene Assay kit (Roche, Cat. # 1814036). Around five stable clones with highly stimulated luciferase activities by 10 nM NDP-αMSH are established.

### EXAMPLE 2

### Melanocortin Receptor Whole Cell cAMP Accumulation Assay

Hank's Balanced Salt Solution without phenol red (HBSS-092), 1 M HEPES, Dulbecco's Modified Eagle Media (DMEM), Fetal Bovine Serum (FBS), Antibiotic/Antimycotic Solution, and sodium acetate are obtained from GibcoBRL. Triton X-100, ascorbic acid, cAMP, and 3-isobutyl-1-methyl-xanthine (IBMX) are purchased from Sigma. Bovine Serum Albumin (BSA) is obtained from Roche. SPA PVT antibody-binding beads type II anti-sheep beads and ¹²⁵I cAMP are obtained from Amersham. Anti-goat cAMP antibody is obtained from ICN. Enzyme Free Cell Dissociation Solution Hank's based is obtained from Specialty Media. NDP-αMSH is obtained from Calbiochem. Dimethylsulfoxide (DMSO) is obtained from Aldrich.

Compound Preparation

In the agonist assay, compounds are prepared as 10 mM and NDP-αMSH (control) as 33.3 μM stock solutions in 100% DMSO. These solutions are serially diluted in 100% DMSO. The compound plate is further diluted in compound dilution buffer (HBSS-092, 1 mM Ascorbic Acid, 1 mM IBMX, 0.6% DMSO, 0.1% BSA) to yield a final concentration range in the assay between 600 nM-6 pM for compound and 100 nM-1 pM for NDP-αMSH control in 0.5% DMSO. Twenty μL of compound solution are transferred from this plate into four PET 96-well plates (all assays are performed in duplicate for each receptor).

Cell Culture and Cell Stimulation

HEK 293 cells stably transfected with the human MC3R or MC4R are grown in DMEM containing 10% FBS and 1% Antibiotic/Antimycotic Solution. On the day of the assay, the cells are dislodged with enzyme free cell dissociation solution and re-suspended in cell buffer (HBSS-092, 0.1% BSA, 10 mM HEPES) at 1 ×10⁶ cells/mL. Forty μL of cell suspension are added per well to PET 96-well plates containing 20 μL of diluted compound or control. Plates are incubated at 37° C. in a waterbath for 20 minutes. The assay is stopped by adding 50 μL Quench Buffer (50 mM sodium acetate, 0.25% Triton X-100).

Determination of cAMP Concentrations

Radioligand binding assays are run in SPA buffer (50 mM sodium acetate, 0.1% BSA). The beads, antibody, and radioligand are diluted in SPA buffer to provide sufficient volume for each 96-well plate. To each quenched assay well is added 100 μL cocktail containing 33.33 μL of beads, 33.33 μL antibody, and 33.33 μL ¹²⁵I-cAMP. This is based on a final concentration of 6.3 mg/mL beads, 0.65% anti-goat antibody, and 61 MP of ¹²⁵I-cAMP (containing 25,000-30,000 CPM) in a final assay volume of 210 μL. The plates are counted in a Wallac MicroBeta counter after a 12-hour incubation.

The data are converted to pmol of cAMP using a standard curve assayed under the same conditions. The data are analyzed using Activity Base software to generate agonist potencies (EC50) and percent relative efficacy data compared to NDP-αMSH.

TABLE 2
MC3 Potency

| Compound | MC3 | MC4 | MC1/MC4 |
|---|---|---|---|
| No. | Kᵢ (nM) | Kᵢ (nM) | selectivity |
| 1 | 500.00 | 127.80 | 3.91 |
| 2 | 125.22 | 0.39 | 10.70 |
| 3 | 34.86 | 0.41 | 4.00 |
| 4 | 29.73 | 0.23 | 0.26 |
| 5 | 47.98 | 0.42 | 5.00 |
| 6 | 295.77 | 2.15 | 35.74 |
| 7 | 41.63 | 0.82 | 15.00 |
| 8 | 27.63 | 1.43 | 3.33 |
| 9 | 364.26 | 2.39 | 10.00 |
| 10 | 18.60 | 0.10 | 9.50 |
| 11 | 125.88 | 1.26 | 11.00 |
| 12 | 121.56 | 1.10 | 6.72 |
| 13 | 31.61 | 0.34 | 10.65 |
| 14 | 32.18 | 0.35 | 12.54 |
| 15 | 82.71 | 0.67 | 14.75 |
| 16 | 302.94 | 0.83 | 2.94 |
| 17 | 26.05 | 0.57 | 10.42 |
| 18 | 23.28 | 0.35 | 8.15 |
| 19 | 27.97 | 0.53 | 7.64 |
| 20 | 54.74 | 0.48 | 4.81 |
| 21 | 38.63 | 0.22 | 10.27 |
| 22 | 30.34 | 0.27 | 6.85 |
| 23 | 12.97 | 0.26 | 10.54 |
| 24 | 23.63 | 0.44 | 8.00 |
| 25 | 36.96 | 0.32 | 11.00 |
| 26 | 58.82 | 0.71 | 38.90 |
| 27 | 79.16 | 1.05 | 30.11 |
| 28 | 71.27 | 1.18 | 26.35 |
| 29 | 500.00 | 3.18 | 15.00 |
| 30 | 418.44 | 2.36 | 38.48 |
| 31 | 103.71 | 0.75 | 57.02 |
| 32 | 17.14 | 0.37 | 66.88 |
| 33 | 10.13 | 0.35 | 79.54 |
| 34 | 500.00 | 43.42 | 11.52 |
| 35 | 28.58 | 1.03 | 1.17 |
| 36 | 127.33 | 1.66 | 1.22 |
| 37 | 316.28 | 1.81 | 36.99 |
| 38 | 319.52 | 2.55 | 28.16 |
| 39 | 233.85 | 2.08 | 19.67 |
| 40 | 62.30 | 0.96 | 25.92 |
| 41 | 62.48 | 0.60 | 58.47 |
| 42 | 43.20 | 0.40 | 44.63 |
| 43 | 157.41 | 1.06 | 11.00 |
| 44 | 106.86 | 0.95 | 15.00 |
| 45 | 500.00 | 3.03 | 30.47 |
| 46 | 65.70 | 0.73 | |
| 47 | >500 | 53.32 | |
| 48 | 29.33 | 0.43 | 26.80 |
| 49 | 500.00 | 3.14 | 35.35 |
| 50 | 2.89 | 0.21 | 36.10 |
| 51 | 428.74 | 6.52 | 76.75 |
| 52 | 66.33 | 0.55 | 30.54 |
| 53 | >500 | 8.68 | |
| 54 | 53.53 | 0.48 | 20.85 |
| 55 | 173.71 | 1.67 | 28.81 |
| 56 | 500.00 | 23.39 | 21.38 |
| 57 | 199.51 | 2.26 | 29.00 |
| 58 | 107.42 | 0.81 | 31.69 |
| 59 | 73.59 | 0.86 | 20.92 |
| 60 | 97.06 | 1.51 | 29.95 |
| 61 | 37.06 | 0.87 | 1.70 |
| 62 | 136.88 | 0.75 | 46.91 |
| 63 | 266.49 | 2.28 | 30.51 |
| 64 | 13.21 | 0.62 | 4.12 |
| 65 | 73.11 | 6.53 | 2.70 |
| 66 | 38.89 | 0.83 | 13.23 |
| 67 | 20.35 | 0.26 | 9.15 |
| 68 | 23.20 | 0.63 | 14.08 |
| 69 | 221.58 | 3.00 | 18.38 |
| 70 | 9.93 | 0.30 | 2.00 |
| 71 | 36.23 | 2.11 | 5.13 |
| 72 | 54.77 | 0.78 | 22.31 |
| 73 | 410.77 | 8.78 | 12.77 |
| 74 | 107.19 | 1.21 | 12.00 |
| 75 | 143.16 | 2.31 | 6.00 |
| 76 | 500.00 | 24.23 | 6.00 |
| 77 | 23.52 | 0.41 | 28.38 |
| 78 | 500.00 | 7.28 | 9.00 |
| 79 | 44.55 | 0.57 | 21.79 |
| 80 | 111.56 | 5.27 | 8.24 |
| 81 | 496.28 | 5.93 | 101.69 |
| 82 | 500.00 | 300.86 | 1.66 |
| 83 | 14.96 | 0.26 | 45.95 |
| 84 | 218.44 | 3.32 | 150.60 |
| 85 | 500.00 | 188.06 | 2.66 |
| 86 | 2.91 | 0.13 | 66.21 |
| 87 | 42.39 | 1.11 | 316.25 |
| 88 | 500.00 | 55.14 | 9.07 |
| 89 | 2.54 | 0.11 | 71.43 |
| 90 | 16.78 | 0.86 | 237.22 |
| 91 | 385.67 | 23.65 | 21.14 |
| 92 | 8.56 | 0.52 | 12.06 |
| 93 | 16.69 | 0.65 | 1.48 |
| 94 | 157.20 | 5.12 | 16.97 |
| 95 | 500.00 | 155.83 | 3.21 |
| 96 | 85.30 | 4.01 | 20.87 |
| 97 | 17.90 | 0.58 | 8.03 |
| 98 | 253.73 | 11.54 | 7.43 |
| 99 | 209.62 | 5.66 | 88.42 |
| 100 | 500.00 | 300.24 | 1.67 |
| 101 | 26.39 | 14.00 | 0.97 |
| 102 | 500.00 | 105.01 | 4.76 |
| 103 | 20.26 | 6.62 | 75.59 |
| 104 | 500.00 | 135.91 | 3.68 |
| 105 | 500.00 | 20.80 | 24.04 |
| 106 | 319.65 | 20.88 | 23.95 |
| 107 | 500.00 | 500.00 | 1.00 |
| 108 | 166.58 | 31.36 | 5.99 |
| 109 | 332.51 | 82.70 | 6.05 |
| 110 | 500 | 117.22 | 4.27 |
| 111 | 500.00 | 65.19 | 7.67 |
| 112 | 500.00 | 88.97 | 5.62 |
| 113 | 260.27 | 37.01 | 13.51 |
| 114 | 46.33 | 1.35 | 4.00 |
| 115 | 39.77 | 1.15 | 2.00 |
| 116 | 69.30 | 2.00 | 4.00 |
| 117 | 14.57 | 0.63 | 1.00 |
| 118 | 126.67 | 4.59 | 4.52 |
| 119 | 16.13 | 0.57 | 0.86 |
| 120 | 13.49 | 0.40 | 1.00 |
| 121 | 4.08 | 0.34 | 0.74 |
| 122 | 3.96 | 0.30 | 0.90 |
| 123 | 13.37 | 1.13 | 2.42 |
| 124 | 329.20 | 2.36 | 18.11 |
| 125 | 500.00 | 19.94 | 25.08 |
| 126 | 91.03 | 0.74 | 22.64 |
| 127 | 16.91 | 0.28 | 20.25 |
| 128 | 97.97 | 0.89 | 22.46 |
| 129 | 272.56 | 2.18 | 22.16 |
| 130 | 228.60 | 1.98 | 26.88 |
| 131 | 500.00 | 11.18 | 7.00 |
| 132 | 43.44 | 0.34 | 77.32 |
| 133 | | 9.08 | 31.29 |
| 134 | 7.19 | 0.13 | 68.42 |
| 135 | 1.02 | 0.06 | 120.27 |
| 136 | >500 | 55.30 | 7.01 |
| 137 | 9.77 | 0.32 | 54.60 |
| 138 | 112.13 | 3.08 | 38.81 |
| 139 | 8.46 | 0.38 | 44.29 |
| 140 | 1.87 | 0.20 | 128.15 |
| 141 | 3.69 | 0.19 | 83.00 |
| 142 | 1.96 | 0.11 | 35.55 |
| 143 | 0.60 | 0.08 | 19.27 |
| 144 | 2.23 | 0.30 | 14.85 |
| 145 | 5.79 | 0.73 | 3.82 |
| 146 | 3.28 | 0.32 | 27.43 |
| 147 | 0.65 | 0.07 | 0.86 |
| 148 | 2.86 | 0.10 | 51.98 |
| 149 | 1.01 | 0.07 | 51.85 |
| 150 | 36.88 | 2.35 | 12.88 |
| 151 | 41.43 | 4.35 | 14.00 |
| 152 | 42.42 | 1.77 | 7.73 |
| 153 | 4.11 | 0.10 | 41.81 |
| 154 | | 0.21 | 36.00 |
| 155 | 23.60 | 0.68 | 55.84 |
| 156 | 35.13 | 1.31 | 158.41 |
| 157 | 354.85 | 28.42 | 17.60 |
| 158 | 1.91 | 0.08 | 50.25 |
| 159 | 19.64 | 0.74 | 49.41 |
| 160 | 0.28 | 0.05 | 0.90 |
| 161 | 1.67 | 0.08 | 2.18 |
| 162 | 2.79 | 0.08 | 30.07 |
| 163 | 85.82 | 2.28 | 19.46 |
| 164 | 9.04 | 0.38 | 7.79 |
| 165 | 63.75 | 1.45 | 13.53 |
| 166 | 500.00 | 25.05 | 9.38 |
| 167 | 500.00 | 93.07 | 3.36 |
| 168 | 64.37 | 1.35 | 212.71 |
| 169 | 0.86 | 0.03 | 1804.00 |
| 170 | 4.85 | 0.13 | 9.00 |
| 171 | 2.50 | 0.10 | 75.11 |
| 172 | 3.68 | 0.15 | 26.45 |
| 173 | 9.65 | 0.37 | 29.10 |
| 174 | 4.86 | 0.23 | 4.98 |
| 175 | 40.26 | 1.29 | |
| 176 | 19.16 | 0.49 | |
| 177 | 1.24 | 0.05 | |
| 178 | 10.52 | 0.38 | |
| 179 | 500.00 | 93.46 | 5.35 |
| 180 | 500.00 | 16.46 | 30.38 |
| 181 | 500.00 | 6.07 | 45.25 |
| 182 | 70.85 | 0.89 | 185.74 |
| 183 | 360.28 | 9.37 | 53.39 |
| 184 | 128.37 | 2.51 | 97.44 |
| 185 | 12.74 | 0.47 | 269.59 |
| 186 | 182.17 | 5.21 | 11.44 |
| 187 | 22.73 | 2.02 | 20.76 |
| 188 | 12.55 | 0.92 | 29.56 |
| 189 | 90.86 | 2.72 | 23.31 |
| 190 | 33.03 | 0.17 | 367.10 |
| 191 | 4.13 | 0.26 | 127.33 |
| 192 | 500.00 | 36.70 | 1.00 |
| 193 | 95.41 | 2.59 | 26.59 |
| 194 | 41.86 | 2.93 | 10.61 |
| 195 | 14.14 | 0.87 | 32.56 |
| 196 | 85.08 | 2.10 | 4.98 |
| 197 | 247.79 | 21.81 | 1.00 |
| 198 | 500.00 | 16.72 | 13.07 |

### EXAMPLE 3

### Administration of an MC3R Agonist Peptide to MC4R knockout and MC4R Wild Type Mice

To establish the effect of MC3R on feeding and weight loss, an MC3R agonist peptide is injected into two groups: one containing MC4R knockout mice and another containing MC4R wild type mice. For comparison, a saline vehicle, used for the MC3R agonist peptide formulation for delivery, is also injected into two similar groups of mice. The administration is shown in Table 3, below.

TABLE 3
Groups of mice and samples administered.

| | | | | Dose |
|---|---|---|---|---|
| Group | Mouse Type | Substance | Delivery Method | (mg/kg) |
| 1 | MC4R WT* | Saline | subcutaneous injection | 0 |
| 2 | MC4R k/o* | Saline | subcutaneous injection | 0 |
| 3 | MC4R WT | MC3R agonist | subcutaneous injection | 40 |
| 4 | MC4R k/o | MC3R agonist | subcutaneous injection | 40 |

*“WT” stands for “wild type” and “k/o” stands for “knockout”

The mice are weighed before and after the experiment to establish any change in mass. The MC3R agonist peptide injections are made at time zero. The mice are then placed into chambers of an open-circuit calorimeter (Oxymax, Columbus, Ohio) for twenty-four hours. Oxygen consumption and carbon dioxide release are recorded sequentially, with each animal being measured approximately every 39 minutes. Pre-weighed pelletized food (Teklad TD95217) is placed into the chambers with the mice, and the food is reweighed at the end of the study to determine food intake. All data are entered into the calorimeter program, and calculations for fuel intake, fuel utilization, energy balance, respiratory quotient, caloric expenditure, and fat utilization are calculated. Results from a typical experiment are shown in Table 4, below.

TABLE 4
Calorimetry data.

| Parameter Measured | Group 1 | Group 2 | Group 3 | Group 4 |
|---|---|---|---|---|
| Fuel Intake [kcal/kg/24 hr] | 428.48 | 302.49 | 76.7 | 270.32 |
| Fuel Utilization [kcal/kg/24 hr] | 299.6 | 268.31 | 318.6 | 267.93 |
| Energy Balance [kcal/kg/24 hr] | 128.9 | 34.18 | −242.2 | 2.39 |
| Respiratory Quotient | 0.85 | 0.82 | 0.702 | 0.793 |
| Caloric Expenditure | 12.2 | 11.07 | 12.96 | 10.93 |
| [average kcal/kg/hr] | | | | |
| Fat Utilization [kcal/kg/24 hr] | 110.61 | 127.25 | 284.91 | 156.65 |

The peptide in this experiment is an MC3R/MC4R agonist peptide (MC3R, Ki=54.77 nM; MC4R, Ki=0.78 nM). To differentiate between activity of one receptor and the other, MC4R knockout and wild type mice are used. Since the agonist potency is lower at MC3R, high doses of the agonist were administered to the MC4R knockout and wild type mice to induce and differentiate an MC3R-mediated metabolic response.

The ratio of VCO2/VO2 (CO2 produced during substrate oxidation/oxygen consumed to oxidized substrate) measured in indirect calorimetry is the respiratory quotient (“RQ”). RQ is indicative of the type of fuel substrate being utilized. RQ values of 1.0 indicate carbohydrate utilization, while decreased RQ values near 0.7 indicate a switch to utilization of fat as fuel substrate. Looking at the data, saline treated Groups 1 and 2 exhibit similar RQ values to each other. The twenty-four hour average RQ value for Group 3 decreased, indicating a switch from carbohydrate utilization to fat utilization. Group 4 exhibited a less significant decrease in RQ; however, this change in RQ is measurable in animals lacking a function MC4 receptor, indicating induction of a MC3 receptor-mediated response.

Twenty-four hour energy balance and fuel intake are also affected by agonism of the MC3 receptor. With respect to energy balance, Groups 1 and 2 have quite different values; thus, the best comparison is between the Groups 1 and 3 (the two wild type groups) and Groups 2 and 4 (the two knockout groups). Comparing Groups 1 and 3, agonism of the MC4 receptor causes a change in energy balance. Fuel intake (kcal/kg/24 hr) is significantly decreased for group 3, accompanied by an increase in fuel utilization (kcal/kg/24 hr), resulting in a large negative energy balance. While not as large as the change in Group 3, Group 4 also exhibits a decrease in fuel intake, compared to group 2, with no change in utilization. These results indicate subtle changes in fuel intake and utilization attributed solely to MC3R metabolic effect in MC4R knockout animals.

These data demonstrate that agonism of the MC3 receptor can lead to changes in energy balance and fat utilization. Such results suggest that MC3R agonist peptides will be useful in the treatment of metabolic disorders such as obesity, diabetes mellitus, and dyslipidemias.

1-26. (canceled)

27. A method of treating a metabolic disorder, comprising administering an effective amount of an MC3R agonist peptide to a patient in need thereof, wherein the MC3R agonist peptide comprises a peptide selected from the group consisting of Compound Numbers 1-198, with the proviso that peptides having Compound Numbers 1, 29, 34, 45, 47, 49, 53, 56, 76, 78, 82, 85, 88, 95, 100, 102, 104, 105, 107, 110, 111, 112, 125, 131, 136, 166, 167, 179, 180, 181, 192, and 198 are excluded.

28. The method of claim 27, wherein the MC3R agonist peptide comprises a peptide selected from the group consisting of Compound Numbers 50, 86, 89, 92, 121, 122, 134, 135, 137, 141 through 149, 153, 158, 160, 161, 162, 164, 169 through 174, 177, and 191.

29. The method of claim 28, wherein the MC3R agonist peptide comprises a peptide selected from the group consisting of cyclo[hCys-His-(4-Cl-D-Phe)-Arg-Trp-Cys]-NH₂, N-cyclopentanecarbonyl-cyclo[hCys-His-D-Phe-Arg-Trp-Cys]-NH₂, 4-phenylbutyryl-cyclo[hCys-His-D-Phe-Arg-Trp-Cys]-NH₂, 5-guanidinovaleryl-cyclo[hCys-His-D-Phe-Arg-Trp-Cys]-NH₂, Ac-nLeu-cyclo[hCys-His-D-Phe-Arg-Trp-Cys]-NH₂, phenylsulfonyl-Gly-cyclo[hCys-His-D-Phe-Arg-Trp-Cys]-NH₂, or Ac-cyclo[hCys-His-(4-Cl-D-Phe)-Arg-Trp-penicillamine]-NH₂.

30. The method of claim 29, wherein the MC3R agonist peptide is 5-guanidinovaleryl-cyclo[hCys-His-D-Phe-Arg-Trp-Cys]-NH₂.

31. The method of claim 29, wherein the MC3R agonist peptide is Ac-nLeu-cyclo[hCys-His-D-Phe-Arg-Trp-Cys]-NH₂.

32. The method of claim 28, wherein the MC3R agonist peptide is 3-guanidinopropionyl-cyclo[hCys-His-D-Phe-Arg-Trp-Cys]-NH₂.

33. The method of claim 27, wherein the metabolic disorder is obesity.

34. The method of claim 27, wherein the metabolic disorder is diabetes mellitus.

35. The method of claim 27, wherein the metabolic disorder is cachexia.

36. The method of claim 27, wherein the metabolic disorder is sarcopenia.

37. The method of claim 27, wherein the metabolic disorder is a cardiovascular disorder.

38. A method of inducing weight loss in a patient, comprising administering an effective amount of an MC3R agonist peptide to a patient in need thereof, wherein the MC3R agonist peptide comprises a peptide selected from the group consisting of Compound Numbers 1-198, with the proviso that peptides having Compound Numbers 1, 29, 34, 45, 47, 49, 53, 56, 76, 78, 82, 85, 88, 95, 100, 102, 104, 105, 107, 110, 111, 112, 125, 131, 136, 166, 167, 179, 180, 181, 192, and 198 are excluded.

39. The method of claim 38, wherein the MC3R agonist peptide is 5-guanidinovaleryl-cyclo [hCys-His-D-Phe-Arg-Trp-Cys]-NH₂.

40. The method of claim 38, wherein the MC3R agonist peptide is Ac-nLeu-cyclo[hCys-His-D-Phe-Arg-Trp-Cys]-NH_{2 .}

41. A method of increasing muscle mass in a patient, comprising administering an effective amount of an MC3R agonist peptide to a patient in need thereof, wherein the MC3R agonist peptide comprises a peptide selected from the group consisting of Compound Numbers 1-198, with the proviso that peptides having Compound Numbers 1, 29, 34, 45, 47, 49, 53, 56, 76, 78, 82, 85, 88, 95, 100, 102, 104, 105, 107, 110, 111, 112, 125, 131, 136, 166, 167, 179, 180, 181, 192, and 198 are excluded.

42. The method of claim 41, wherein the MC3R agonist peptide is 5-guanidinovaleryl-cyclo[hCys-His-D-Phe-Arg-Trp-Cys]-NH₂.

43. The method of claim 41, wherein the MC3R agonist peptide is Ac-nLeu-cyclo[hCys-His-D-Phe-Arg-Tr-Cys]-NH₂.
